(19) United States
(12) Patent Application Publication
(10) Pub. No.: US 2026/0027191 A1
(43) Pub. Date: Jan. 29, 2026

(54) COMPOSITIONS AND METHODS FOR TREATING BACTERIAL DISEASE

(71) Applicant: Arizona Board of Regents on Behalf of the University of Arizona, Tucson, AZ (US)

(72) Inventors: Magdalene So, Tucson, AZ (US); Maria A. Rendon-Espinosa, Tucson, AZ (US)

(21) Appl. No.: 18/856,809

(22) PCT Filed: Apr. 14, 2023
(86) PCT No.: PCT/US2023/065791

§ 371 Date: Oct. 14, 2024

Related U.S. Application Data
(60) Provisional application No. 63/331,399, filed on Apr. 15, 2022.

(51) Int. Cl. A61K 38/45; A61P 31/04

(52) U.S. Cl. CPC A61K 38/45; A61P 31/04

(57) ABSTRACT

The present invention relates to compositions and methods for preventing and/or treating bacterial disease (e.g., disease caused by Neisseria sp. such as Neisseria gonorrhoeae or Neisseria meningitidis). In particular, the present invention provides compositions comprising an effective amount of a nucleic acid, wherein such compositions are capable of killing or inhibiting the growth of a Neisseria sp.

### STATEMENT REGARDING RELATED APPLICATIONS

The present application claims priority to U.S. Provisional Patent Application No. 63/331,399, filed Apr. 15, 2022, which is hereby incorporated by reference in its entirety.

### STATEMENT REGARDING FEDERALLY SPONSORED RESEARCH OR DEVELOPMENT

This invention was made with government support under Grant No. AI151117 awarded by National Institutes of Health. The government has certain rights in the invention.

### SEQUENCE LISTING

The text of the computer readable sequence listing filed herewith, titled “40819_252_SequenceListing_Corrected.xml”, created Oct. 16, 2025, having a file size of 32,911 bytes, is hereby incorporated by reference in its entirety.

### FIELD OF THE INVENTION

The present invention relates to compositions and methods for preventing and/or treating bacterial disease (e.g., disease caused by Neisseria sp. such as Neisseria gonorrhoeae or Neisseria meningitidis). In particular, the present invention provides compositions comprising an effective amount of a nucleic acid, wherein such compositions are capable of killing or inhibiting the growth of a Neisseria sp.

### BACKGROUND

Neisseria gonorrhoeae (Ngo) and Neisseria meningitidis (Nme) are pathogens that cause high impact diseases in humans. Ngo infects the urinary tract and oropharynx of males and females. Ngo causes over 160 million new infections each year, worldwide. There is currently no vaccine against Ngo. Ngo has developed resistance to all antibiotics used for its treatment, leading the NIH, CDC and WHO to place Ngo on their list of “superbugs”. NIH has announced initiatives to accelerate the development of novel antimicrobials and identify new targets for antimicrobials and antibiotics against these superbugs.

Nme colonizes the upper respiratory tract, entering the bloodstream to cause septicemia and crossing the blood-brain barrier to cause meningitis. Crowded living conditions and large migrations encourage the spread of Nme, and are the main cause of epidemics and micro-epidemics around the world. As the CDC does not require the reporting of Nme infections, there is no accurate information on their incidence. Vaccines have significantly reduced the incidence of meningococcal disease in developed countries. However, they do not cover all Nme serogroups and are unaffordable in poor countries. Nme continues to cause occasional epidemics in parts of Africa and the Middle East.

Improved therapeutic options for preventing, and/or treating infection by Neisseria gonorrhoeae and Neisseria meningitidis are needed.

### SUMMARY OF THE INVENTION

Experiments described herein identified discreet pieces of DNA whose nucleotide sequences derive from Ngo chromosomal loci and whose nucleotides are methylated differently than those in Ngo DNA. These DNAs are an improvement over N. elongata chromosomal DNA because their sequences are in Ngo and absent or not commonly found from commensal Neisseria, and because of this leave commensal Neisseria unharmed.

Accordingly, in some embodiments, provided herein is a method for the inhibition of bacterial growth and/or for the killing of a target bacterium, comprising the step of adding to the target bacterium a composition comprising an effective amount of a nucleic acid comprising at least one Neisseria-specific DNA uptake sequence (DUS) and a nucleic acid encoding at least one or more Ngo DNA methyltransferases selected from NgoAXIV, NgoAl, and NgoAXV, thereby inhibiting bacterial growth and/or killing the bacteria. These nucleic acid sequences can span the entire gene or a portion of the genes in question (e.g., 10, 50, 100, 1000, 2000, or 5000 nucleotides up to the entire gene).

Further embodiments provide a composition comprising a nucleic acid comprising at least one Neisseria-specific DNA uptake sequence (DUS) and a nucleic acid encoding at least a portion (e.g., 10, 50, 100, 1000, 2000, or 5000 nucleotides up to the entire gene) of one or more Ngo DNA methyltransferases selected from NgoAXIV, NgoAI, or NgoAXV, and a pharmaceutically acceptable carrier.

Yet other embodiments provide the use of a composition comprising a nucleic acid comprising at least one Neisseria-specific DNA uptake sequence (DUS) and a nucleic acid encoding at least a portion (e.g., 10, 50, 100, 1000, 2000, or 5000 nucleotides up to the entire gene) of one or more Ngo DNA methyltransferases selected from NgoAXIV, NgoAl, or NgoAXV, and a pharmaceutically acceptable carrier to inhibit bacterial growth and/or kill a target bacterium.

Certain embodiments provide a composition comprising a nucleic acid comprising at least one Neisseria-specific DNA uptake sequence (DUS) and a nucleic acid encoding at least a portion (e.g., 10, 50, 100, 1000, 2000, or 5000 nucleotides up to the entire gene) of one or more of the following Ngo genes: tdfF, tdfH, or iga, and a pharmaceutically acceptable carrier and methods of using such a composition to inhibit bacterial growth and/or kill a target bacterium or the use of the composition to inhibit bacterial growth and/or kill a target bacterium.

The present disclosure is not limited to particular nucleic acids. Examples include but are not limited to plasmid DNA, bacterial artificial chromosome DNA, or genomic DNA. In some embodiments, the one or more nucleic acids contain sequences within one, two or all three of the following loci: ngoAXIV, ngoAl, and ngoAXV (e.g., present as a concatemer). In some embodiments, the one or more nucleic acids contain sequences within one, two or all three of the following loci: tdfF, tdfH, and iga (e.g., present as a concatemer). These nucleic acid sequences can span the entire gene or a portion of the genes. In some embodiments, the nucleic acid is produced by another organism, e.g. E. coli, or synthesized in vitro.

The present disclosure is not limited to a particular DUS. Examples include but are not limited to the sequence N₁N₂ N₃N₄N₅N₆N₇CTGN₈A (SEQ ID NO:1), wherein N₁ is A or T. N₂ is T. G, or A, N₃ is G or C, N₄ is C or T, N₅ is C, T, or A, N₆ is G or A, N₇ is T or C, and No is C or A (e.g., A [T/G] GCCGTCTGAA (SEQ ID NO:2) or GCCGTCTGAA (SEQ ID NO:3)).

In some embodiments, the composition is a pharmaceutical composition (e.g., a personal lubricant). In some embodiments, the target bacterium is Neisseria gonorrhoeae (Ngo) or Neisseria meningitidis (Nme). In some embodiments, the composition is administered topically. In some embodiments, the composition does not kill and/or inhibit the growth of commensal strains of Neisseria.

Additional embodiments are described herein.

### BRIEF DESCRIPTION OF THE DRAWINGS

FIG. 1 shows exemplary nucleic acid sequences described herein.

FIG. 2 shows a schematic of an exemplary killing assay.

FIG. 3 shows that low-passage and multidrug resistance Ngo isolates are susceptible to killing by Nel DNA.

FIG. 4 shows a map of the location of Ngo genes described herein.

FIG. 5 shows killing of Neisseria gonorrhoeae strain MS11 by DNA sequences. A. The names and diagram of the nine sequences selected for testing as a microbicide. B. Killing efficiency of the DNA molecules in (A). Bars represent the average of at least 3 biological replicates. Statistical analysis was done using one way ANOVA with Tukey's Post Test correction using GraphPad Prism 5.0 software. ns, not significant.

FIG. 6 shows commensal Neisseria lactamica, a member of the microbiota, is not killed by DNA constructs.

FIG. 7 shows that Ngo MS11 is killed more effectively when Nel DNA is suspended in 1% hydroxyethyl cellulose, a common ingredient in lubricants.

FIG. 8 shows that MB 14-1-15 efficiently kills all tested Neisseria gonorrhoeae isolates, whether dissolved in buffer (10 mM Tris) or the personal lubricants KY Jelly and Astroglide. Abbreviations: MB, MB 14-1-15 DNA; KY, KY jelly; AG, Astroglide. * P<0.01; **, P<0.1; ***, P<0.001; ****, P<0.0001; ns, not significant.

FIG. 9 shows that MB 14-1-15 efficiently kills Neisseria meningitidis isolate 8013. Abbreviations: MB, MB 14-1-15 DNA. * P≤0.01; ***, P<0.001; ****, P<0.0001.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “subject” and “patient” refer to any animal, such as a mammal or other animal, for example, a dog, cat, bird, livestock, and preferably a human.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “administration” refers to the act of giving a drug, prodrug, antibody, vaccine, or other agent, or therapeutic treatment to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Coadministration” refers to administration of more than one chemical agent or therapeutic treatment to a physiological system (e.g., a subject or in vivo, in vitro, or ex vivo cells, tissues, and organs). As used herein, administration “in combination with” one or more further therapeutic agents includes simultaneous (concurrent) and consecutive administration in any order. “Coadministration” of therapeutic treatments may be concurrent, or in any temporal order or physical combination.

As used herein, “carriers” include pharmaceutically acceptable carriers, excipients, or stabilizers which are nontoxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; antioxidants including ascorbic acid; low molecular weight (less than about 10 residues) polypeptides: proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

As used herein, the term “nucleic acid molecule” refers to any nucleic acid containing molecule, including but not limited to, DNA or RNA. The nucleic acid molecule may comprise one or more nucleotides. The term encompasses sequences that include any of the known base analogs of DNA and RNA including, but not limited to, 4-acetylcytosine, 8-hydroxy-N₆-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N₆-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N₆-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N₆-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example, a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

An “effective amount” is an amount sufficient to effect beneficial or desired results. An effective amount can be administered in one or more administrations.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues (e.g., kidney tissue or cells), and gases. Biological samples include blood products, such as plasma, serum and the like. Such examples are not however to be construed as limiting the sample types applicable to the present disclosure.

### DETAILED DESCRIPTION

Nme and Ngo descended from a commensal Neisseria ancestor and are genetically related to present day commensal Neisseria spp (Quillin, S. J. and H. S. Seifert, Neisseria gonorrhoeae host adaptation and pathogenesis. Nat Rev Microbiol, 2018. 16 (4): p. 226-240). This shared ancestry with commensals supports that their tendency to cause persistent asymptomatic infection may be governed by a shared repertoire of host interaction factors. Indeed, of the approximately 177 Ngo and Nme genes previously reported to encode host interaction factors, 69 are conserved in all 19 other Neisseria spp. evaluated (Marri, P. R., et al., Genome sequencing reveals widespread virulence gene exchange among human Neisseria species. PLOS One, 2010. 5 (7): p. e11835). While genes unique to Ngo and Nme undoubtedly contribute to pathogenesis, the importance of the shared host interaction factors to infection cannot be discounted. Moreover, these shared host interaction factors may also contribute to the ability of commensal Neisseria to sporadically cause disease.

Taking advantage of the fact that pathogenic Neisseria have numerous genes in common with commensal Neisseria, experiments described herein resulted in the development of an antimicrobial that kills Ngo and Nme. DNA from commensal Neisseria kills Ngo and Nme if the DNA is taken up by the pathogen, the DNA attempts to recombine with the many homologous sequences in the pathogen chromosome, and the DNA is methylated differently than Ngo DNA so that it is recognized and cleaved by Ngo restriction enzymes and the damage done to the chromosome by enzyme cleavage leads to cell death (Kim, W. J., et al., Cell Host & Microbe, 2019, 26, 1-12 PMCID: PMC6728082).

U.S. Pat. No. 10,286,016, Compositions and Methods for Treating Gonorrhea, describes the use of DNA from Neisseria elongata, a commensal, as a microbicidal agent for preventing Ngo infection. N. elongata DNA kills Ngo in vitro and in vivo. In fact, any DNA is able to kill the pathogen, providing it is methylated differently than Ngo DNA, has sequence homology to this pathogen's chromosome, and is taken up (internalized) by the pathogen (Kim, W. J., Higashi, D., Goytia, M., Rendon, M. A., Pilligua-Lucas, M., Bronnimann, M., McLean, J. A., Duncan, J., Trees, D., Jerse, A. E. and So, M. (2019) Commensal Neisseria Kill Neisseria gonorrhoeae through a DNA-Dependent Mechanism. Cell Host & Microbe 26, 1-12. PMCID: PMC6728082).

The present disclosure expands upon this observation by providing specific Neisseria spp. sequences that are able to kill pathogenic strains of Neisseria.

Experiments described herein demonstrated that DNA molecules kill Ngo if they contain sequences with homology to the Ngo genome and a Neisseria-specific DNA Uptake Sequence (DUS), and if their nucleotides are methylated differently than Ngo DNA. For example, DNA molecules containing sequences unique to Ngo, such as those in the tdfF, tdfH, and iga loci, and DNA molecules containing sequences within Ngo DNA methylase genes, kill the pathogen, provided they contain a DUS, have homology to Ngo chromosomal DNA, and is methylated differently than Ngo DNA. In addition, a concatemer of Ngo DNA methylase genes, MB14-1-5, kills Ngo when dissolved in buffer and suspended in personal lubricants, making it possible to apply a microbicide such as MB14-1-5 topically.

Accordingly, in some embodiments, provided herein is a method for the inhibition of bacterial growth and/or for the killing of a target bacterium, comprising the step of adding to the target bacterium a composition comprising an effective amount of a nucleic acid comprising at least one Neisseria-specific DNA uptake sequence (DUS) and a nucleic acid encoding one or more Ngo DNA methyltransferases selected from NgoAXIV, NgoAI, or NgoAXV, thereby inhibiting bacterial growth and/or killing the bacteria.

Further embodiments provide a composition comprising a nucleic acid comprising at least one Neisseria-specific DNA uptake sequence (DUS) and a nucleic acid encoding one or more Ngo DNA methyltransferases selected from NgoAXIV, NgoAl, or NgoAXV, and a pharmaceutically acceptable carrier.

Yet other embodiments provide the use of a composition comprising a nucleic acid comprising at least one Neisseria-specific DNA uptake sequence (DUS) and a nucleic acid encoding one or more Ngo DNA methyltransferases selected from NgoAXIV, NgoAl, or

NgoAXV, and a pharmaceutically acceptable carrier to inhibit bacterial growth and/or kill a target bacterium. These nucleic acid sequences can span the entire gene or a portion of the genes in question (e.g., at least 10, 20, 50, 100, 200, 500, 1000, 5000 or more nucleotides up to the entire sequence).

Certain embodiments provide a composition comprising a nucleic acid comprising at least one Neisseria-specific DNA uptake sequence (DUS) and a nucleic acid encoding one or more of the following proteins abundant in Ngo TdfF, TdfH, or Iga, and a pharmaceutically acceptable carrier and methods of using such a composition to inhibit bacterial growth and/or kill a target bacterium or the use of the composition to inhibit bacterial growth and/or kill a target bacterium. These nucleic acid sequences can span the entire gene or a portion of the genes in question.

The present disclosure is not limited to particular nucleic acids. Examples include but are not limited to a plasmid, a bacterial artificial chromosome, genomic DNA, or DNA synthesized in vitro. In some embodiments, the one or more nucleic acids are NgoAXIV, NgoAl, and NgoAXV (e.g., present as a concatemer). In some embodiments, the one or more nucleic acids are tdfF, tdfH, and iga (e.g., present as a concatemer).

In some embodiments, the one or more nucleic acids are synthesized in vitro or produced by a different microorganism (e.g., E. coli).

The present invention is not limited to a particular DUS (See e.g., Frye S. A., Nilsen, M., Tønjum, T., Ambu, O. H. Dialects of the DNA uptake sequence in Neisseria. PLOS Genet. Apr;9 (4): e1003458. doi: 10.1371/journal. pgen.1003458. Epub 2013 Apr. 18 (2013).; herein incorporated by reference in its entirety). Examples include but are not limited to the sequence N₁N₂N₃N₄N₅N₆N₇CTGN₈A (SEQ ID NO:1), wherein Ni is A or T, N₂ is T, G, or A, N₃ is G or C. N₄ is C or T, N₅ is C, T, or A, No is G or A, N₇ is T or C, and Ng is C or A (e.g., A [T/G] GCCGTCTGAA (SEQ ID NO:2) or GCCGTCTGAA (SEQ ID NO:3)).

In some embodiments, the composition is a pharmaceutical composition (e.g., a personal lubricant). In some embodiments, the target bacterium is Neisseria gonorrhoeae (Ngo) or Neisseria meningitidis (Nme). In some embodiments, the composition is administered topically. In some embodiments, the composition does not kill and/or inhibit the growth of commensal strains of Neisseria.

In some embodiments, the composition is an antiseptic. Antiseptics are antimicrobial substances that are applied to living tissue/skin to reduce the possibility of infection and/or sepsis, and/or putrefaction. Antiseptics are generally distinguished from antibiotics by their ability to be transported through the lymphatic system to destroy bacteria within the body, and from disinfectants, which destroy microorganisms found on non-living objects. In some embodiments, antiseptic compositions comprising an effective amount of a commensal species of Neisseria (e.g., an effective amount of an extract of a commensal species of Neisseria) (e.g., Nel, Npo) capable of inhibiting the growth of Ngo and/or is killing Ngo are provided. For example, in some embodiments, such an antiseptic composition can be applied to the tissue of a subject (e.g., a human subject) for purposes of preventing the growth or inducing the killing of Ngo.

In some embodiments, the composition is a disinfectant. In some embodiments, disinfectant compositions comprising an effective amount of a commensal species of Neisseria (e.g., an effective amount of an extract of a commensal species of Neisseria) (e.g., Nel, Npo) capable of inhibiting the growth or of inducing the killing of Ngo are provided. For example, in some embodiments, such a disinfectant composition can be used in the cleaning of hospitals such as in cleaning of an operating room and/or surgery equipment. Disinfectants should generally be distinguished from antibiotics that destroy microorganisms within the body, and from antiseptics, which destroy microorganisms on living tissue.

In some embodiments, the compositions are used for anti-fouling. Anti-fouling is the process of removing or inhibiting the accumulation of biofouling. Biofouling or biological fouling is the undesirable accumulation of microorganisms, plants, algae, and animals on surfaces.

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include personal lubricants, transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present disclosure the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present disclosure. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

In some embodiments, the composition is a gel (e.g., formulated for delivery to a mucosal surface). In some embodiments, the gel coats a product for use in treating or preventing infection by Ngo or Nme (e.g., a condom). In some embodiments, the composition stabilizes the nucleic acid from degradation by enzymes in the mucosa.

In some embodiments, the composition is formulated for delivery to the oropharynx (e.g., as a toothpaste or mouthwash).

In some embodiments, the composition is added to a personal lubricant (e.g., water based, silicone based, or oil based). In some embodiments, any one of many commercially available personal lubricants are utilized (e.g., available from Johnson and Johnson, New Brunswick, NJ or Biofilm, Inc, Vista, CA).

In some embodiments, the composition is coated onto a condom (e.g., male or female condom).

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating clinician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

The composition according to the present invention can be co-administered to an individual in need thereof in combination with one or more drugs such as one or more drugs with antibacterial effect. The one or more antibiotics can be selected from the group consisting of Amikacin disulfate salt, Amikacin hydrate, Anisomycin from Streptomyces griseolus, Apramycin sulfate salt, Azithromycin, Blasticidine S hydrochloride, Brefeldin A, Brefeldin A from Penicillium brefeldianum, Butirosin sulfate salt, Butirosin A from Bacillus vitellinus, Chloramphenicol, Chloramphenicol base, Chloramphenicol succinate sodium salt, Chlortetracycline hydrochloride, Chlortetracycline hydrochloride from Streptomyces aureofaciens, Clindamycin 2-phosphate, Clindamycin hydrochloride, Clotrimazole, Cycloheximide from microbial, Demeclocycline hydrochloride, Dibekacin sulfate salt, Dihydrostreptomycin sesquisulfate, Dihydrostreptomycin solution, Doxycycline hyclate, Duramycin from Streptoverticillium cinnamoneus, Emetine dihydrochloride hydrate), Erythromycin, Erythromycin USP, Erythromycin powder, Erythromycin, Temephos, Erythromycin estolate, Erythromycin ethyl succinate, Erythromycin standard solution, Erythromycin stearate, Fusidic acid sodium salt, G 418 disulfate salt, G 418 disulfate salt powder, G 418 disulfate salt solution liquid, Gentamicin solution liquid, Gentamicin solution, Gentamicin sulfate Micromonospora purpurea, Gentamicin sulfate salt, Gentamicin sulfate salt powder USP, Gentamicin-Glutamine solution liquid, Helvolic acid from Cephalosporium caerulens, Hygromycin B Streptomyces hygroscopicus, Hygromycin B Streptomyces hygroscopicus powder, Hygromycin B solution Streptomyces hygroscopicus, Josamycin, Josamycin solution, Kanamycin B sulfate salt, Kanamycin disulfate salt from Streptomyces kanamyceticus, Kanamycin monosulfate from Streptomyces kanamyceticus, Kanamycin monosulfate from Streptomyces kanamyceticus powder USP, Kanamycin solution from Streptomyces kanamyceticus, Kirromycin from Streptomyces collinus, Lincomycin hydrochloride, Lincomycin standard solution, Meclocycline sulfosalicylate salt, Mepartricin, Midecamycin from Streptomyces mycarofaciens, Minocycline hydrochloride crystalline, Neomycin solution, Neomycin trisulfate salt hydrate, Neomycin trisulfate salt hydrate powder, Neomycin trisulfate salt hydrate USP powder, Netilmicin sulfate salt, Nitrofurantoin crystalline, Nourseothricin sulfate, Oleandomycin phosphate salt, Oleandomycin triacetate, Oxytetracycline dihydrate, Oxytetracycline hemicalcium salt, Oxytetracycline hydrochloride, Paromomycin sulfate salt, Puromycin dihydrochloride from Streptomyces alboniger, Rapamycin from Streptomyces hygroscopicus, Ribostamycin sulfate salt, Rifampicin, Rifamycin SV sodium salt, Rosamicin Micromonospora rosaria, Sisomicin sulfate salt, Spectinomycin dihydrochloride hydrate. Spectinomycin dihydrochloride hydrate powder, Spectinomycin dihydrochloride pentahydrate, Spiramycin, Spiramycin from Streptomyces sp., Spiramycin solution, Streptomycin solution, Streptomycin sulfate salt, Streptomycin sulfate salt powder, Tetracycline, Tetracycline hydrochloride, Tetracycline hydrochloride USP, Tetracycline hydrochloride powder, Thiamphenicol, Thiostrepton from Streptomyces azureus, Tobramycin, Tobramycin sulfate salt, Tunicamycin A1 homolog, Tunicamycin C2 homolog, Tunicamycin Streptomyces sp., Tylosin solution, Tylosin tartrate, Viomycin sulfate salt, Virginiamycin M₁, (S)-(+)-Camptothecin, 10-Deacetylbaccatin III from Taxus baccata, 5-Azacytidine, 7-Aminoactinomycin D, 8-Quinolinol crystalline, 8-Quinolinol hemisulfate salt crystalline, 9-Dihydro-13-acetylbaccatin III from Taxus canadensis, Aclarubicin, Aclarubicin hydrochloride, Actinomycin D from Streptomyces sp., Actinomycin I from Streptomyces antibioticus, Actinomycin V from Streptomyces antibioticus, Aphidicolin Nigrospora sphaerica, Bafilomycin A1 from Streptomyces griseus, Bleomycin sulfate from Streptomyces verticillus, Capreomycin sulfate from Streptomyces capreolus, Chromomycin A3 Streptomyces griseus, Cinoxacin, Ciprofloxacin BioChemika, cis-Diammineplatinum (II) dichloride, Coumermycin A1, Cytochalasin B Helminthosporium dematioideum, Cytochalasin D Zygosporium mansonii, Dacarbazine, Daunorubicin hydrochloride, Daunorubicin hydrochloride USP, Distamycin A hydrochloride from Streptomyces distallicus, Doxorubicin hydrochloride, Echinomycin, Echinomycin BioChemika, Enrofloxacin BioChemika, Etoposide, Etoposide solid, Flumequine, Formycin, Fumagillin from Aspergillus fumigatus, Ganciclovir, Gliotoxin from Gliocladium fimbriatum, Lomefloxacin hydrochloride, Metronidazole purum, Mithramycin A from Streptomyces plicatus, Mitomycin C Streptomyces caespitosus, Nalidixic acid, Nalidixic acid sodium salt, Nalidixic acid sodium salt powder, Netropsin dihydrochloride hydrate, Nitrofurantoin, Nogalamycin from Streptomyces nogalater, Nonactin from Streptomyces tsusimaensis, Novobiocin sodium salt, Ofloxacin, Oxolinic acid, Paclitaxel from Taxus yannanensis, Paclitaxel from Taxus brevifolia, Phenazine methosulfate, Phleomycin Streptomyces verticillus, Pipemidic acid, Rebeccamycin from Saccharothrix aerocolonigenes, Sinefungin, Streptonigrin from Streptomyces flocculus, Streptozocin, Succinylsulfathiazole, Sulfadiazine, Sulfadimethoxine, Sulfaguanidine purum, Sulfamethazine, Sulfamonomethoxine, Sulfanilamide, Sulfaquinoxaline sodium salt, Sulfasalazine, Sulfathiazole sodium salt, Trimethoprim, Trimethoprim lactate salt, Tubercidin from Streptomyces tubercidicus, 5-Azacytidine, Cordycepin, Formycin A, (+)-6-Aminopenicillanic acid, 7-Aminodesacetoxycephalosporanic acid, Amoxicillin, Ampicillin, Ampicillin sodium salt, Ampicillin trihydrate, Ampicillin trihydrate USP, Azlocillin sodium salt, Bacitracin Bacillus licheniformis, Bacitracin zinc salt Bacillus licheniformis, Carbenicillin disodium salt, Cefaclor, Cefamandole lithium salt, Cefamandole nafate, Cefamandole sodium salt, Cefazolin sodium salt, Cefinetazole sodium salt, Cefoperazone sodium salt, Cefotaxime sodium salt, Cefsulodin sodium salt, Cefsulodin sodium salt hydrate, Ceftriaxone sodium salt, Cephalexin hydrate, Cephalosporin C zinc salt, Cephalothin sodium salt, Cephapirin sodium salt, Cephradine, Cloxacillin sodium salt, Cloxacillin sodium salt monohydrate, D-~ ( ) Penicillamine hydrochloride, D-Cycloserine microbial, D-Cycloserine powder, Dicloxacillin sodium salt monohydrate, D-Penicillamine, Econazole nitrate salt, Ethambutol dihydrochloride, Lysostaphin from Staphylococcus staphylolyticus, Moxalactam sodium salt, Nafcillin sodium salt monohydrate, Nikkomycin, Nikkomycin Z Streptomyces tendae, Nitrofurantoin crystalline, Oxacillin sodium salt, Penicillic acid powder, Penicillin G potassium salt, Penicillin G potassium salt powder, Penicillin G potassium salt, Penicillin G sodium salt hydrate powder, Penicillin G sodium salt powder, Penicillin G sodium salt, Phenethicillin potassium salt, Phenoxymethylpenicillinic acid potassium salt, Phosphomycin disodium salt, Pipemidic acid, Piperacillin sodium salt, Ristomycin monosulfate, Vancomycin hydrochloride from Streptomyces orientalis, 2-Mercaptopyridine N-oxide sodium salt, 4-Bromocalcimycin A23187 BioChemika, Alamethicin Trichoderma viride, Amphotericin B Streptomyces sp., Amphotericin B preparation, Calcimycin A23187, Calcimycin A23187 hemi (calcium-magnesium) salt, Calcimycin A23187 hemicalcium salt, Calcimycin A23187 hemimagnesium salt, Chlorhexidine diacetate salt monohydrate, Chlorhexidine diacetate salt hydrate, Chlorhexidine digluconate, Clotrimazole, Colistin sodium methanesulfonate, Colistin sodium methanesulfonate from Bacillus colistinus, Colistin sulfate salt, Econazole nitrate salt, Hydrocortisone 21-acetate, Filipin complex Streptomyces filipinensis, Gliotoxin from Gliocladium fimbriatum, Gramicidin A from Bacillus brevis, Gramicidin C from Bacillus brevis, Gramicidin from Bacillus aneurinolyticus (Bacillus brevis), lonomycin calcium salt Streptomyces conglobatus, Lasalocid A sodium salt, Lonomycin A sodium salt from Streptomyces ribosidificus, Monensin sodium salt, N-(6-Aminohexyl)-5-chloro-1-naphthalenesulfonamide hydrochloride, Narasin from Streptomyces auriofaciens, Nigericin sodium salt from Streptomyces hygroscopicus, Nisin from Streptococcus lactis, Nonactin from Streptomyces sp., Nystatin, Nystatin powder, Phenazine methosulfate, Pimaricin, Pimaricin from Streptomyces chattanoogensis, Polymyxin B solution, Polymyxin B sulfate salt, DL-Penicillamine acetone adduct hydrochloride monohydrate, Polymyxin B sulfate salt powder USP, Praziquantel, Salinomycin from Streptomyces albus, Salinomycin from Streptomyces albus, Surfactin from Bacillus subtilis, Valinomycin, (+)-Usnic acid from Usnea dasypoga, (+)-Miconazole nitrate salt, (S)-(+)-Camptothecin, 1-Deoxymannojirimycin hydrochloride, 1-Deoxynojirimycin hydrochloride, 2-Heptyl-4-hydroxyquinoline N-oxide, Cordycepin, 1,10-Phenanthroline hydrochloride monohydrate puriss., 6-Diazo-5-oxo-L-norleucine, 8-Quinolinol crystalline, 8-Quinolinol hemisulfate salt, Antimycin A from Streptomyces sp., Antimycin A1, Antimycin A2, Antimycin A3, Antipain, Ascomycin, Azaserine, Bafilomycin A1 from Streptomyces griseus, Bafilomycin B1 from Streptomyces species, Cerulenin BioChemika, Chloroquine diphosphate salt, Cinoxacin, Ciprofloxacin, Mevastatin BioChemika, Concanamycin A, Concanamycin A Streptomyces sp, Concanamycin C from Streptomyces species, Coumermycin A1, Cyclosporin A from Tolypocladium inflatum, Cyclosporin A, Econazole nitrate salt, Enrofloxacin, Etoposide, Flumequine, Formycin A, Furazolidone, Fusaric acid from Gibberella fujikuroi, Geldanamycin from Streptomyces hygroscopicus, Gliotoxin from Gliocladium fimbriatum, Gramicidin A from Bacillus brevis, Gramicidin C from Bacillus brevis, Gramicidin from Bacillus aneurinolyticus (Bacillus brevis), Gramicidin from Bacillus brevis, Herbimycin A from Streptomyces hygroscopicus, Indomethacin, Irgasan, Lomefloxacin hydrochloride, Mycophenolic acid powder, Myxothiazol BioChemika, N-(6-Aminohexyl)-5-chloro-1-naphthalenesulfonamide hydrochloride, Nalidixic acid, Netropsin dihydrochloride hydrate, Niclosamide, Nikkomycin BioChemika, Nikkomycin Z Streptomyces tendae, N-Methyl-1-deoxynojirimycin, Nogalamycin from Streptomyces nogalater, Nonactin 80% from Streptomyces tsusimaensis, Nonactin from Streptomyces sp., Novobiocin sodium salt, Ofloxacin, Oleandomycin triacetate, Oligomycin Streptomyces diastatochromogenes, Oligomycin A. Oligomycin B, Oligomycin C, Oligomycin Streptomyces diastatochromogenes, Oxolinic acid, Piericidin A from Streptomyces mobaraensis, Pipemidic acid, Radicicol from Diheterospora chlamydosporia solid, Rapamycin from Streptomyces hygroscopicus, Rebeccamycin from Saccharothrix aerocolonigenes, Sinefungin, Staurosporine Streptomyces sp., Stigmatellin, Succinylsulfathiazole, Sulfadiazine, Sulfadimethoxine, Sulfaguanidine purum, Sulfamethazine, Sulfamonomethoxine, Sulfanilamide, Sulfaquinoxaline sodium salt, Sulfasalazine, Sulfathiazole sodium salt, Triacsin C from Streptomyces sp., Trimethoprim, Trimethoprim lactate salt, Vineomycin A1 from Streptomyces albogriseolus subsp., Tectorigenin, and Paracelsin Trichoderma reesei.

In a further embodiment the present invention relates to a kit of parts comprising the composition according to the present invention. The kit of parts comprises at least one additional component, such as instructions for use, and/or one or more drugs for coadministration.

Such compositions are not limited to particular uses. In some embodiments, the compositions are capable of a static action wherein Ngo or Nme growth is inhibited. In some embodiments, the compositions are capable of a cidal action wherein Ngo or Nme organisms are killed. In some embodiments, the compositions are capable of a lytic action wherein Ngo or Nme organisms are killed and lysed.

### EXPERIMENTAL

### Example 1

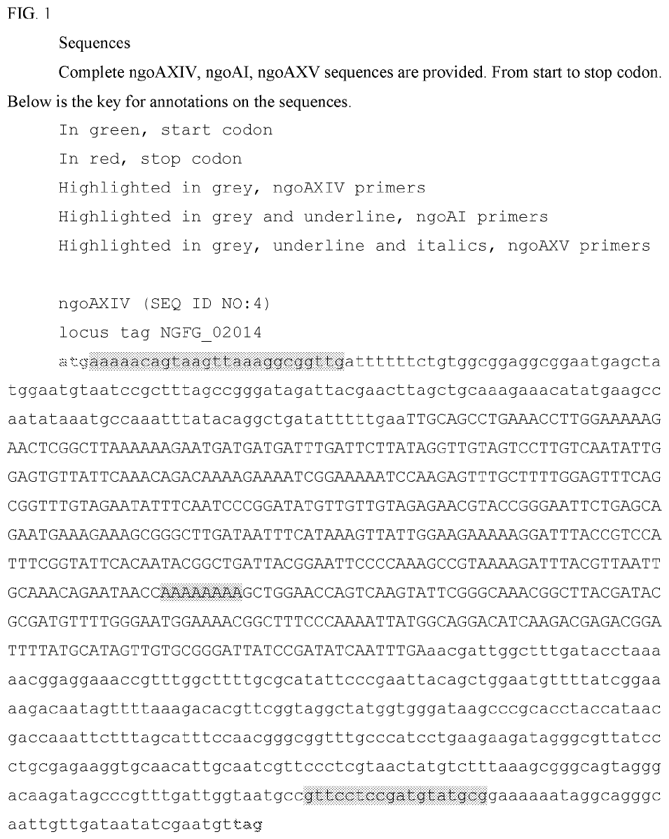

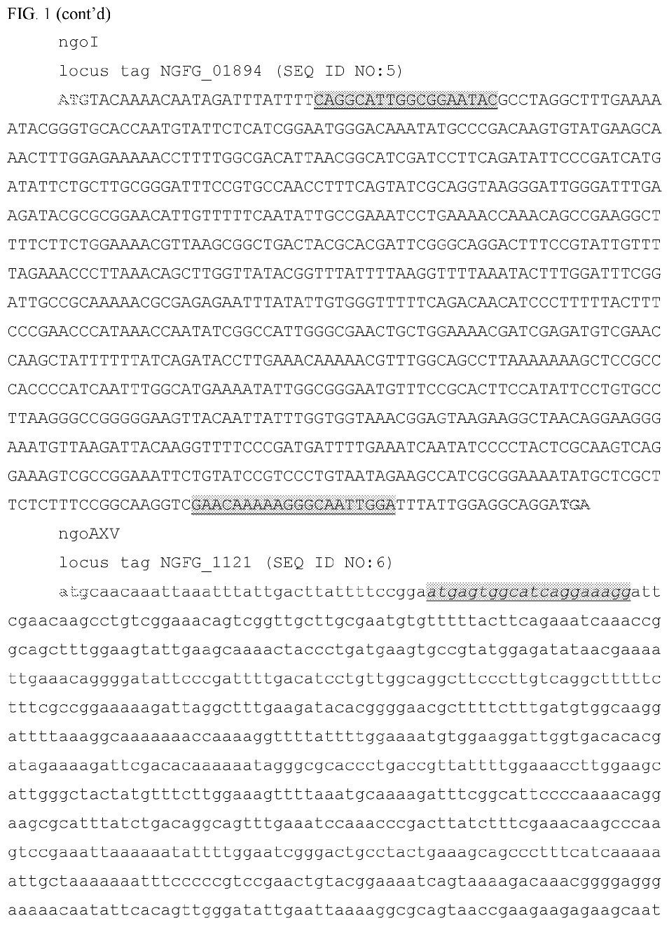

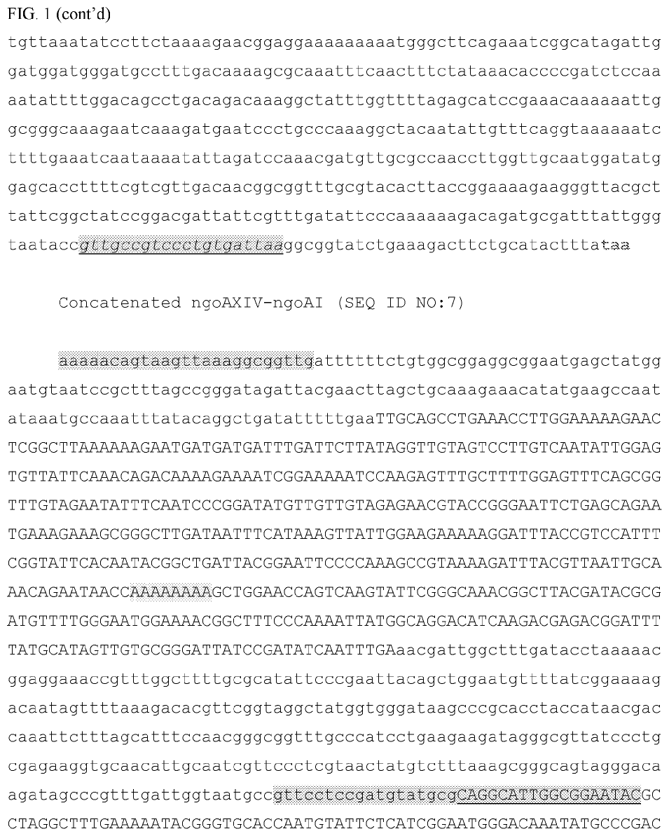

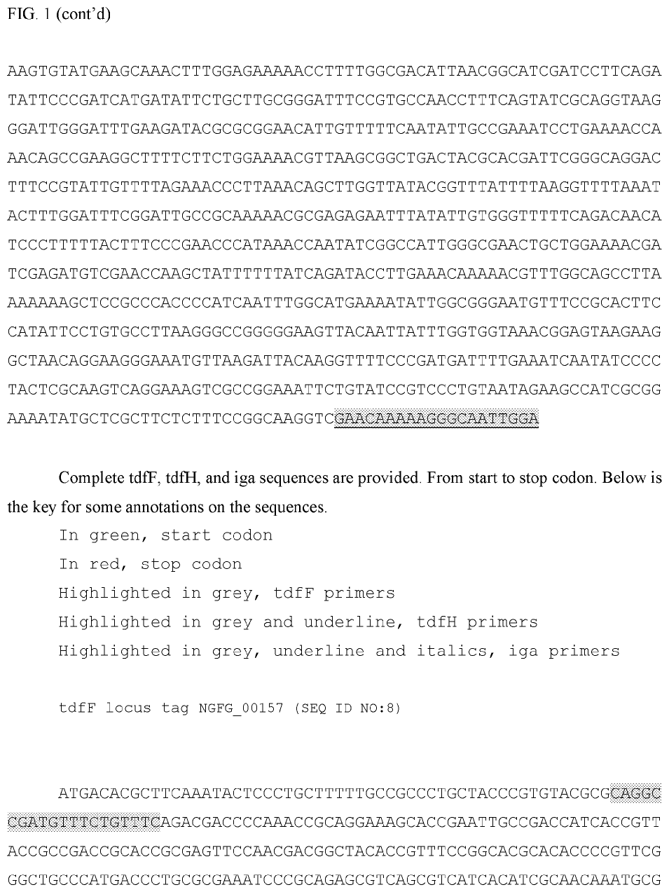

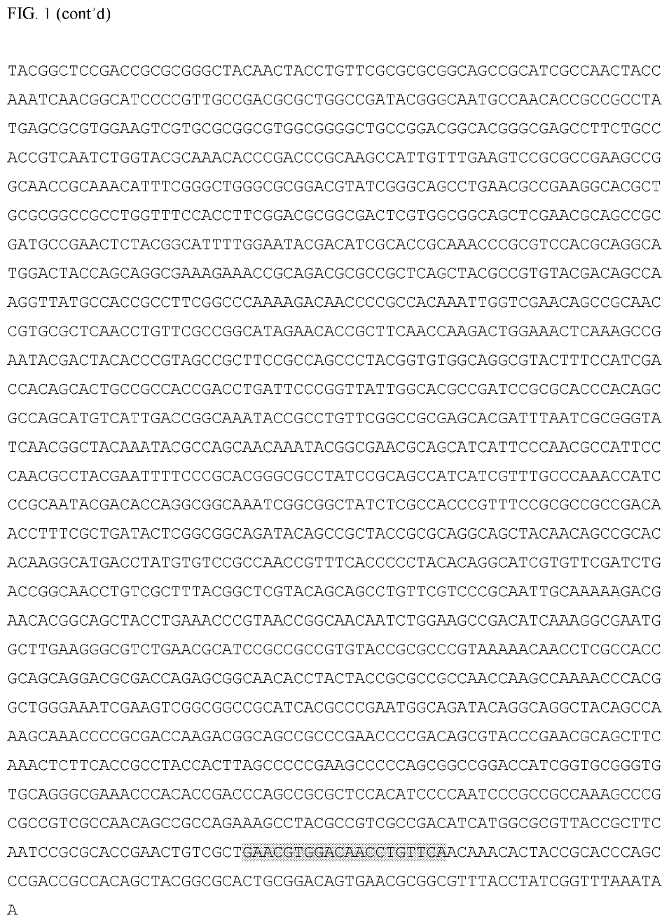

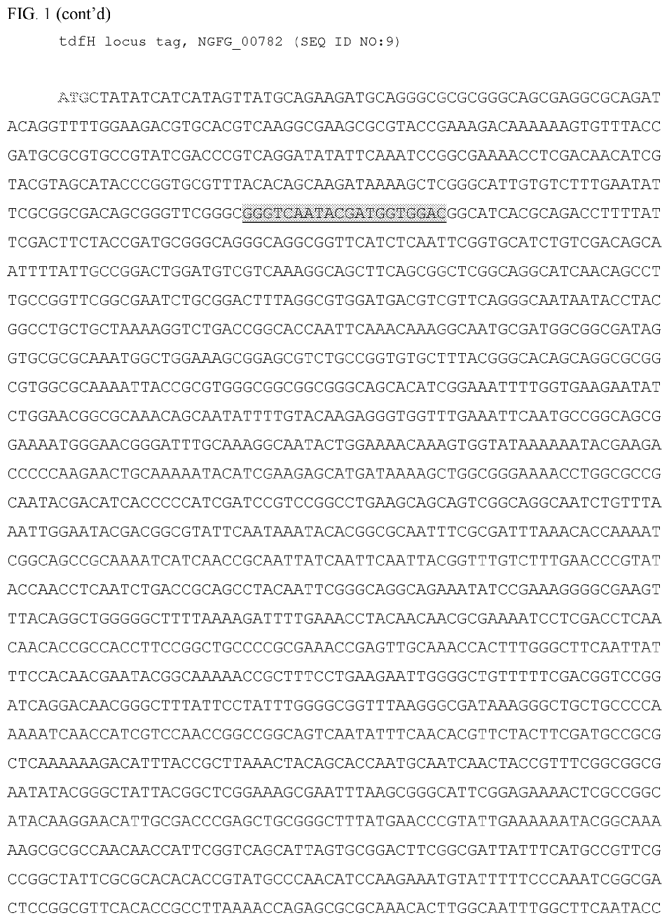

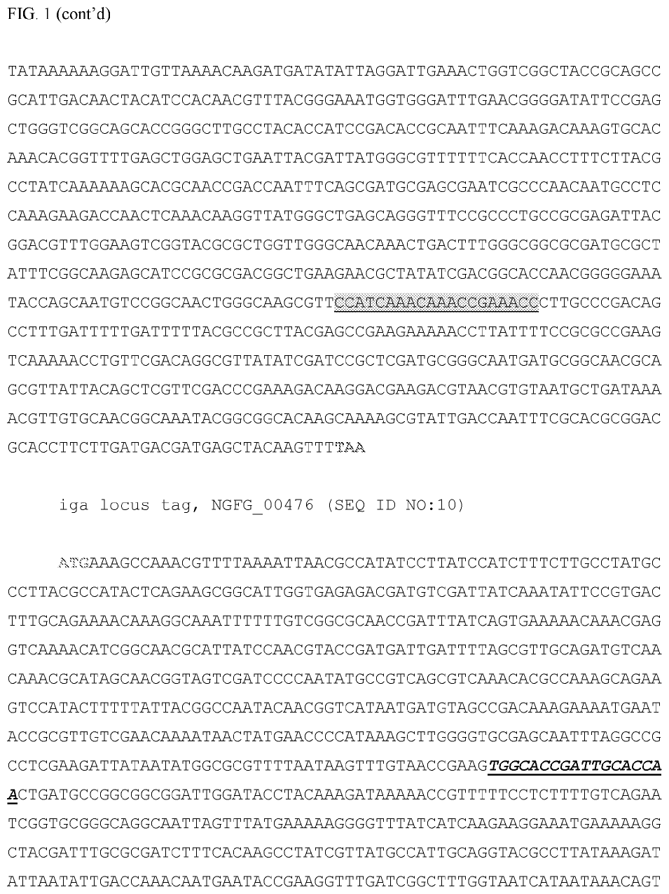

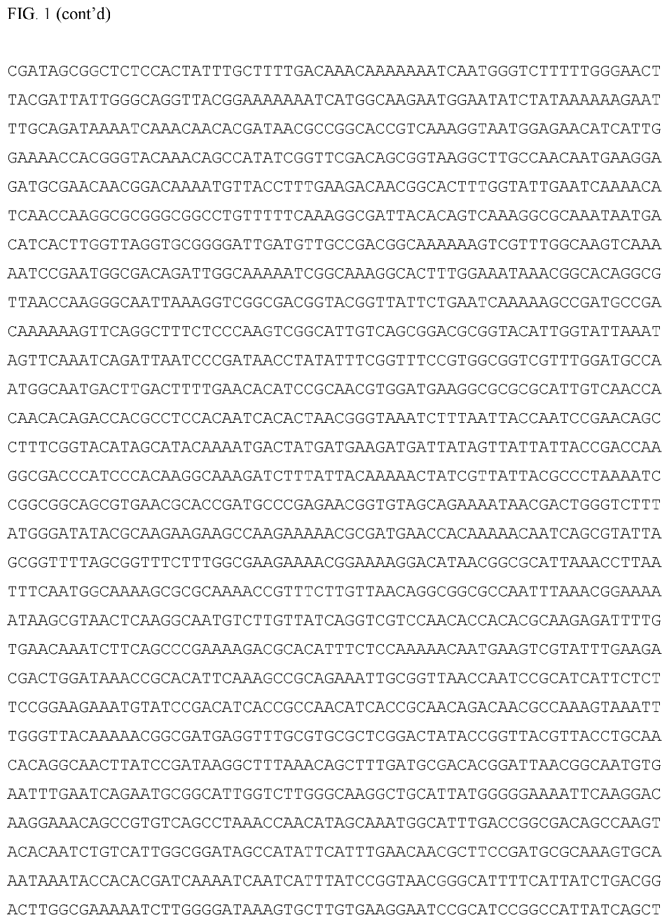

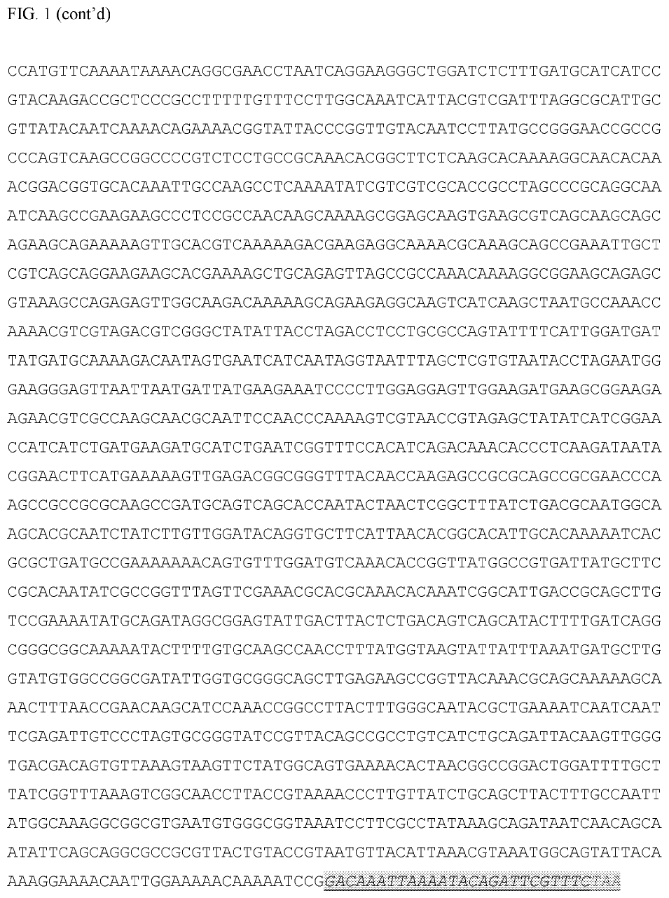

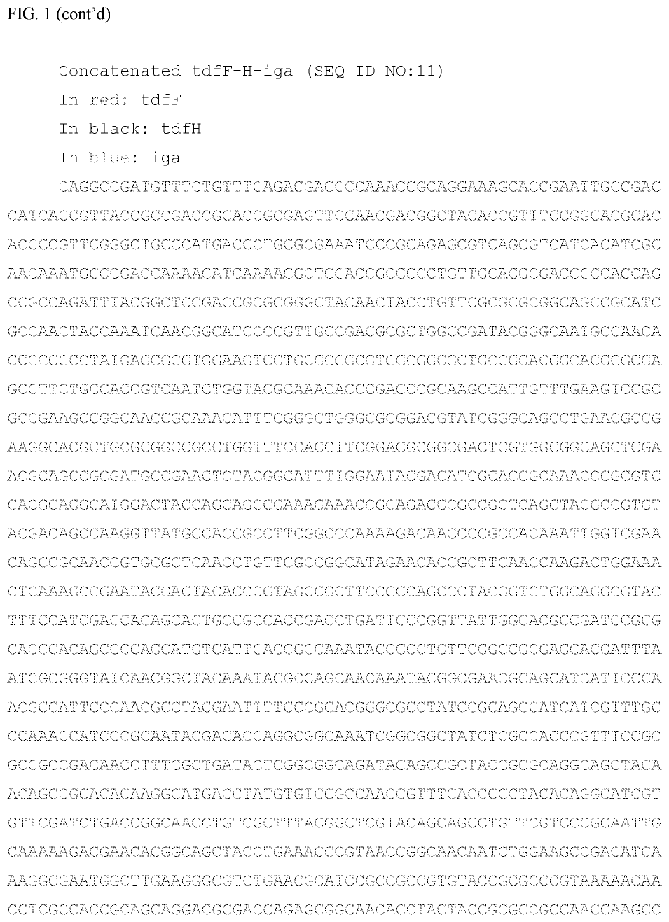

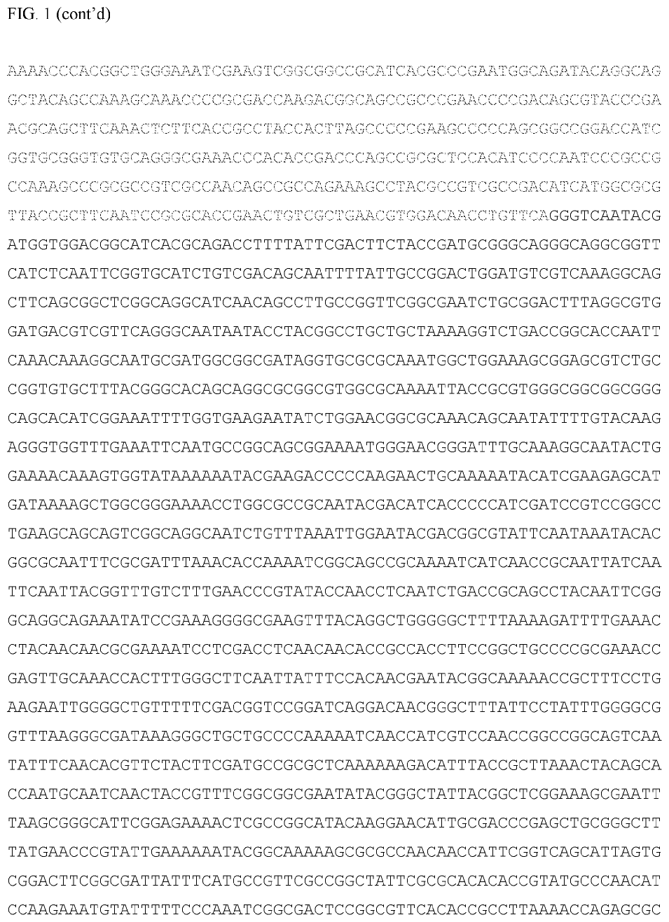

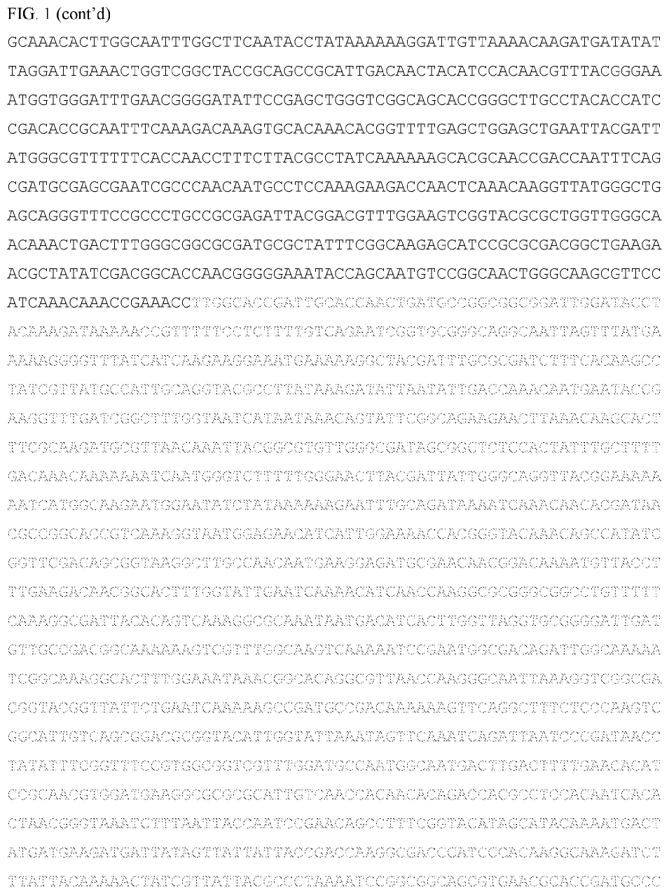

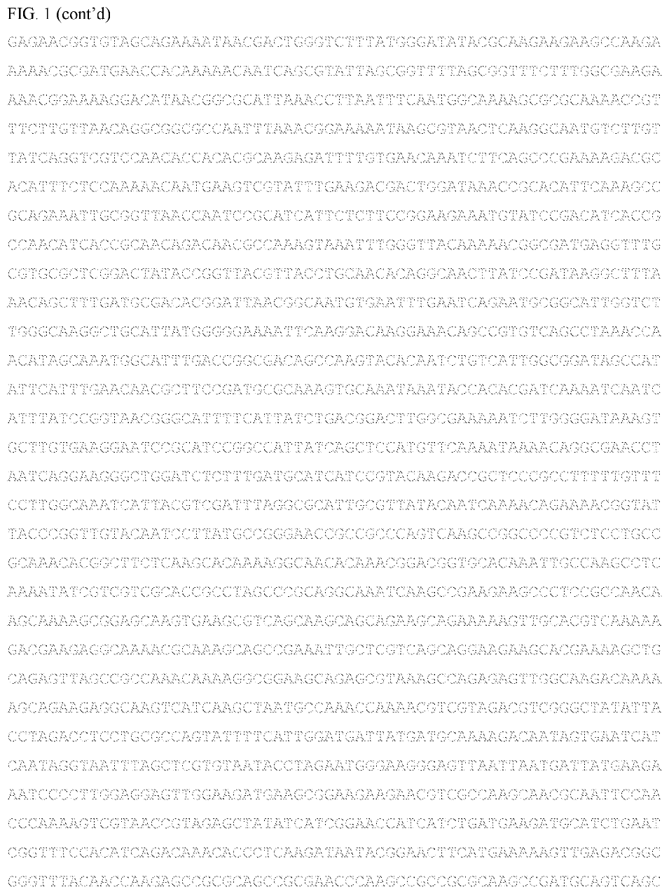

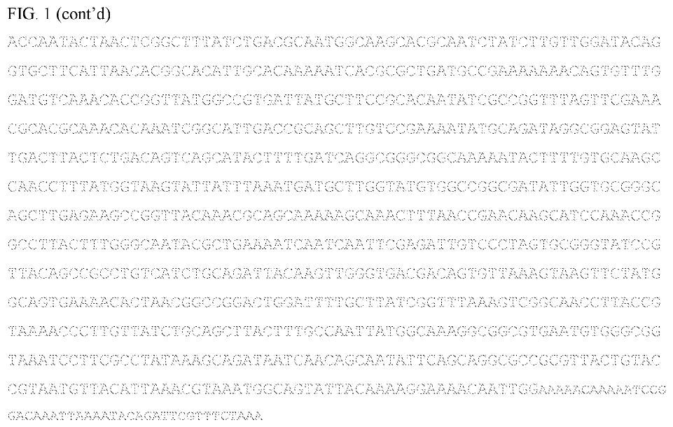

The following Ngo chromosomal sequences were cloned into a plasmid together with the Neisseria-specific DNA Uptake Sequence (DUS) and transformed into E. coli (FIG. 1): (1) internal segments of the tdfF, tdfH, and iga genes, and (2) internal segments of Ngo DNA methylase genes ngoAXIV, ngoAl, and ngoAXV (methylase nomenclature according to (7)). tdfF, tdfH, and iga were selected for testing because they are present in Ngo but rarely in other commensal Neisseria species, and as such are less likely to kill these commensals, which are members of the microbiota. ngoAXIV, ngoAI, and ngoAXV loci were tested because the function of the enzymes encoded by these loci is methylation of cytosines in Ngo DNA, and some of these DNA methylases are essential for Ngo viability.

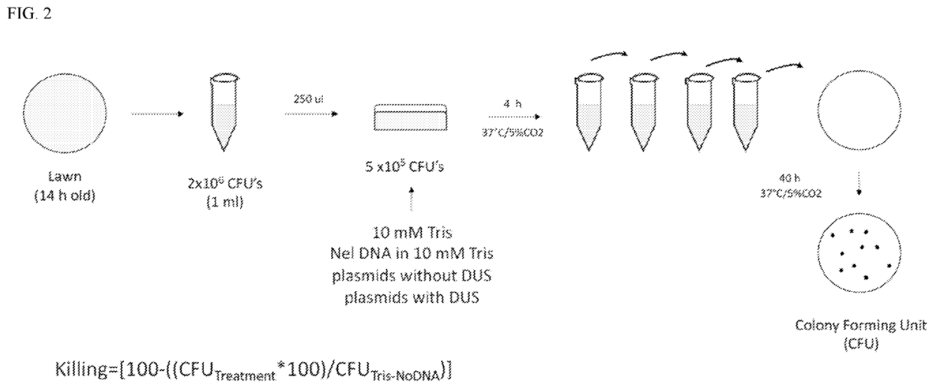

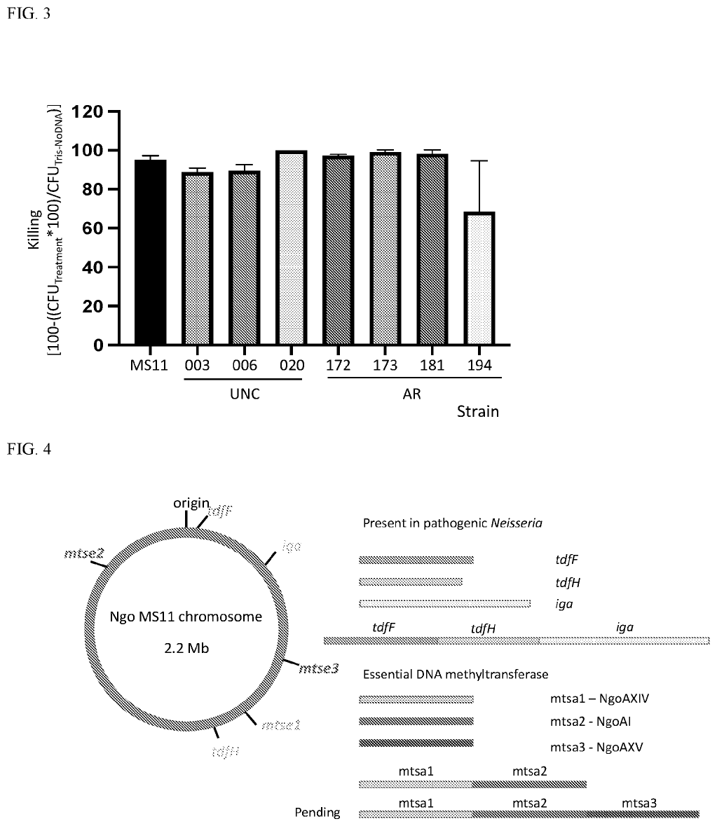

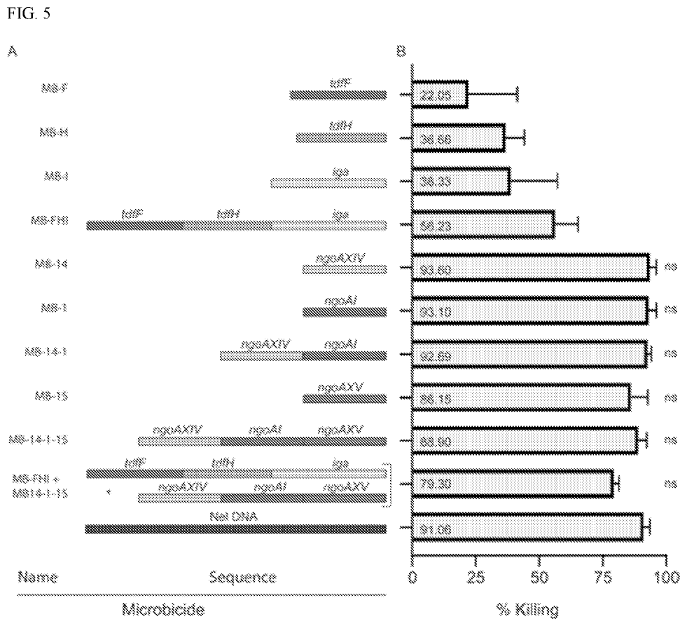

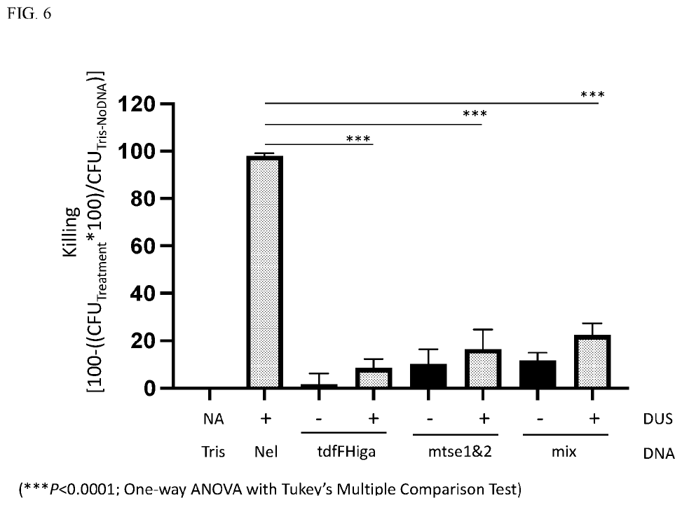

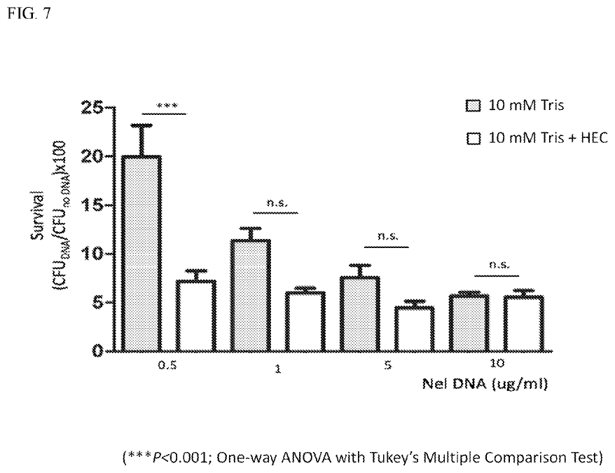

The plasmid DNAs were purified from E. coli and used in an assay (FIG. 2) to assess their ability to kill Ngo strain MS11. The killing efficacy of the tdfF, tdfH, and iga DNAs, alone or concatenated into a single molecule, ranged from 21 to 70% (FIG. 5). The killing efficacy of the ngoAXIV, ngoAl, and ngoAXV DNAs, alone or concatenated, was equal to that of the positive control, Nel DNA, ≥90% (FIG. 5). As reported (6) and U.S. Pat. No. 10,286,016 B2, these DNA sequences must contain the Neisseria-specific DNA uptake sequence (DUS) in order to kill. Concatenates tdfFHiga, ngoAXIV & ngoAl and a mix of the two concatenates did not kill commensal Neisseria lactamica, a member of the human microbiota (FIG. 6).

In conclusion, DNA molecules containing sequences unique to Ngo, such as those in the tdfF, tdfH, and iga loci, and DNA molecules containing sequences within Ngo DNA methylase genes, kill the pathogen, provided they contain a DUS, have homology to Ngo chromosomal DNA, and is methylated differently than Ngo DNA. The killing efficiency of these DNA molecules varies depending on the sequence, with the methylase sequences performing the best. These DNAs do not kill commensal N. lactamica.

### Example 2

The DNA containing all 3 ngoAXI, ngoAXIV and ngoAXV sequences, MB 14-1-15, was selected for further testing. MB 14-1-15 DNA was compared to Nel chromosomal DNA for its effect on human commensal Neisseria species and two bacteria commonly found in the female reproductive tract (Table 1). Assays were conducted as described in Example 1. The killing efficiency of each DNA was calculated as the number of viable bacteria in the DNA-treated sample divided by the number of viable bacteria in the buffer-treated sample (10 mM Tris), expressed as a percentage. None of the tested isolates was harmed by MB 14-1-15, while Neisseria lactamica and Neisseria subflava were mildly susceptible to killing by Nel DNA.

TABLE 1
Commensal bacteria are unharmed by MB 14-1-15 DNA.

| | % Killing, | % Killing, |
|---|---|---|
| | Nel DNA | MB 14-1-15 |
| | (SD, P-value) | (SD, P-value) |
| Commensal Neisseria | | |
| Neisseria elongata ATCC | 16.67 (±10.02, ns) | 11.27 (±11.49, ns) |
| 9315 | | |
| Neisseria subflava NJ9703 | 34.83 (±8.37, 0.03) | 0.06 (±1.4, ns) |
| Neisseria polysaccharea | 6.9 (±7.9, ns) | −0.5* (±7.4, ns) |
| ATCC 43768 | | |
| Neisseria lactamica ATCC | 70.81 (±20.49, 0.04) | −1.05* (1.86, ns) |
| 23970 | | |
| Vaginal bacteria | | |
| Lactobacillus crispatus JV- | 5.93 (±12.95, ns) | 8.6 (±3.68, ns) |
| V01 | | |
| Gardnerella vaginalis 8151B | 6.73 (±6.9, ns) | 8.45 (±8, ns) |

Abbreviations: SD, standard deviation; ns, not significant; nd, not done.
P-value, Tris vs DNA treatment.
*A negative value indicates that the bacteria grew more than the negative control.

For MB 14-1-5 DNA to be an effective anti-Neisseria gonorrhoeae microbicide, it should kill other Neisseria gonorrhoeae strains. One convenient application of MB 14-1-5 is to incorporate it in a commercially available lubricant that can be applied prior to sexual intercourse. Towards these ends, MB 14-1-15 DNA was incorporated into personal lubricants KY Jelly and Astroglide and tested for its ability to kill a number of Ngo clinical isolates (FIG. 8). The assay used in these experiments is described in Example 1. Nel chromosomal DNA was used as the positive control. The negative controls were KY Jelly and Astroglide alone. UNC16 and UNC20 are two low passage clinical isolates, AR 174 is an antibiotic resistant isolate, and MS11 is the lab strain.

KY Jelly and Astroglide alone had little negative effect on MS11 viability (killing 22.92%, ±6 and 21.07%, ±9, respectively). KY Jelly alone had a mild negative effect on UNC16 (27% killing, ±13) and UNC20 (34.35% killing, ±38), but it reduced AR174 viability by 79.97%, ±15. Compared to KY Jelly alone, Astroglide alone had a lesser negative effect on UNC16 (10% killing, ±15) and UNC20 (15% killing, ±7); it did not kill AR174. Nel and MB 14-1-15 DNA killed all the tested Ngo isolates with the same efficiency, whether dissolved in Tris or suspended in either personal lubricant.

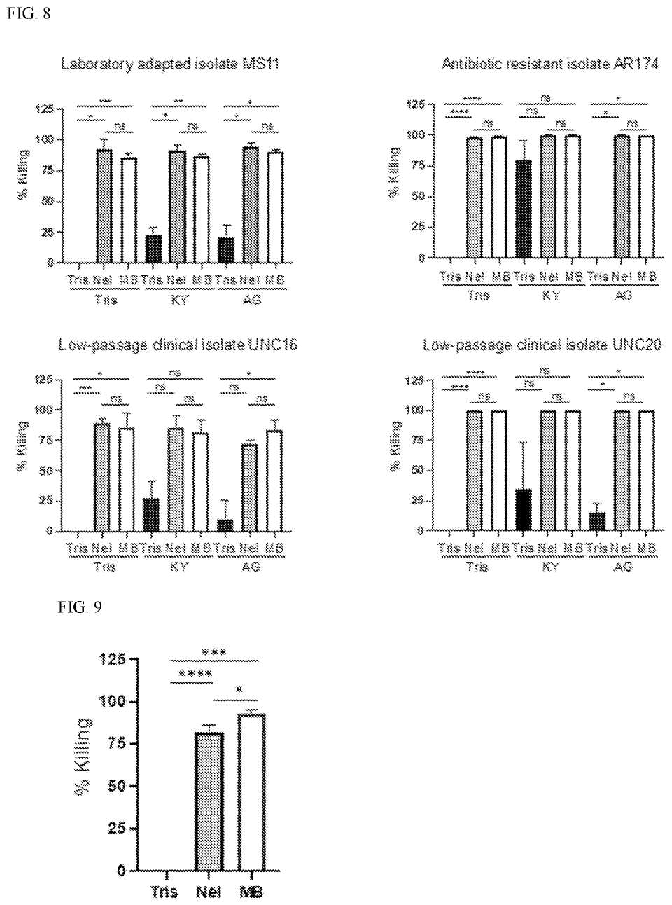

Further experiments demonstrated that MB 14-1-15 efficiently kills Neisseria meningitidis isolate 8013 (FIG. 9).

To summarize, this Example demonstrates that DNA molecules kill Ngo if they contain sequences with homology to the Ngo genome and a Neisseria-specific DNA Uptake Sequence (DUS), and if their nucleotides are methylated differently than Ngo DNA. The DNA molecule tested in the experiments, MB14-1-5, kills Ngo when dissolved in buffer and suspended in personal lubricants, making it possible to apply a microbicide such as MB 14-1-5 topically. DNA molecules lethal to Ngo, which are rare in commensal Neisseria, such as MB 14-1-5, are also good microbicides as they do not harm commensal Neisseria common in the human mucosa.

### REFERENCES CITED

1. Kreisel, KM, Spicknall, IH, Gargano, J W, et al., Sexually transmitted infections among US women and men: prevalence and incidence estimates, 2018. Sex Tranm Dis 48, 208-214 (2021).
2. Janda, W., Bohnhoff, M., Morello, J. & Lerner, S. Prevalence and Site-Pathogen Studies of Neisseria meningitidis and N. gonorrhoeae in Homosexual Men. JAMA 244, 2060-2064 (1980).
3. Gerbase, A., Stein, C., Levison, J. & Htun, Y. Global burden of sexually transmitted diseass (excluding HIV) in the year 2000. Sexually Transmitted Diseases 15-08-06 (2000).
4. Malott, RJ, Keller, BO, Gaudet, R G, et al., (2013) Neisseria gonorrhoeae-derived heptose elicits an innate immune response and drives HIV-1 expression. PNAS, 110:10234-10239.
5. Rice, P. A., Shafer, W. M., Ram, S. & Jerse, A. E. Neisseria gonorrhoeae: Drug Resistance, Mouse Models, and Vaccine Development. Annu Rev Microbiol 71, 665-686, doi: 10.1146/annurev-micro-090816-093530 (2017).
6. Kim, W. J., Higashi, D., Goytia, M., Rendon, M. A., Pilligua-Lucas, M., Bronnimann, M., McLean, J. A., Duncan, J., Trees, D., Jerse, A. E. and So, M. (2019) Commensal Neisseria Kill Neisseria gonorrhoeae through a DNA-Dependent Mechanism. Cell Host & Microbe 26, 1-12. PMCID: PMC6728082.
7. Sanchez-Buso, L, Golparian, D, Parkhill, J, Unemo, M and Harris, SR (2019) Genetic variation regulates the activation and specificity of Restriction-Modification systems in Neisseria gonorrhoeae. Sci Rep 9:14685

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference 10 herein in their entirety.

1. A method for the inhibition of bacterial growth and/or for the killing of a target bacterium, comprising the step of adding to the target bacterium a composition comprising an effective amount of a nucleic acid comprising at least one DNA uptake sequence (DUS) and a nucleic acid encoding at least a portion of one or more Ngo DNA methyltransferases selected from the group consisting of ngoAXIV, ngoAl, and ngoAXV, thereby inhibiting bacterial growth and/or killing said bacteria.

2. The method of claim 1, wherein said nucleic acid is selected from the group consisting of a plasmid, a bacterial artificial chromosome, and genomic DNA.

3. The method of claim 1, wherein said one or more nucleic acids is ngoAXIV, nNgoAl, and goAXV.

4. The method of claim 3, wherein said ngoAXIV, ngoAI, and ngoAXV nucleic acids are present as a concatemer.

5. The method of claim 1, wherein said DUS has the sequence N₁N₂N₃N₄N₅N₆N₇CTGN₈A (SEQ ID NO:1), wherein N₁ is A or T, N₂ is T, G, or A, N₃ is G or C, N₄ is C or T, N₅ is C, T, or A, N₆ is G or A, N₇ is T or C, and N₈ is C or A.

6. The method of claim 5, wherein said DUS has the sequence
(SEQ ID NO: 2)
A[T/G]GCCGTCTGAA
or
(SEQ ID NO: 3)
GCCGTCTGAA.

7. The method of claim 1,
wherein said composition is a pharmaceutical composition.

8. The method of claim 7, wherein said composition is a personal lubricant.

9. The method of claim 1, wherein the target bacterium is Neisseria gonorrhoeae (Ngo) or Neisseria meningitidis (Nme).

10. The method of claim 1, wherein said composition is administered topically.

11. The method of claim 1, wherein said composition does not kill and/or inhibit the growth of commensal species of Neisseria.

12. A composition comprising a nucleic acid comprising at least one DNA uptake sequence (DUS) and a nucleic acid encoding one or more Ngo DNA methyltransferases selected from the group consisting of NgoAXIV, NgoAl, and NgoAXV, and a pharmaceutically acceptable carrier.

13. The composition of claim 12, wherein said nucleic acid is selected from the group consisting of a plasmid, a bacterial artificial chromosome, and genomic DNA.

14. The composition of claim 12, wherein said one or more nucleic acids is ngoAXIV, ngoAl, and ngoAXV.

15. The composition of claim 14, wherein said ngoAXIV, ngoAl, and ngoAXV nucleic acids are present as a concatemer.

16. The composition of claim 12, wherein said DUS has the sequence N₁N₂N₃N₄N₅N₆N₇CTGN₈A (SEQ ID NO:1), wherein N₁ is A or T, N₂ is T, G, or A, N₃ is G or C, N₄ is C or T, N₅ is C, T, or A, N₆ is G or A, N₇ is T or C, and N₈ is C or A.

17. The composition of claim 16, wherein said DUS has the sequence
(SEQ ID NO: 2)
A[T/G]GCCGTCTGAA
or
(SEQ ID NO: 3)
GCCGTCTGAA.

18. The composition of claim 12, wherein said composition is a pharmaceutical composition.

19. The composition of claim 18, wherein said composition is a personal lubricant.

20. (canceled)

21. A composition comprising a nucleic acid comprising at least one DNA uptake sequence (DUS) and a nucleic acid encoding at least a portion of one or more Ngo DNA genes selected from the group consisting of tdfF, tdfH, and iga, and a pharmaceutically acceptable carrier.

22-23. (canceled)


SEQUENCE LISTING (11 sequences)

SEQ ID NO: 1 - skipped

SEQ ID NO: 2 - DNA, 12 bp, Synthetic construct
  source 1..12: mol_type = other DNA
akgccgtctgaa

SEQ ID NO: 3 - DNA, 10 bp, Synthetic construct
  source 1..10: mol_type = other DNA
gccgtctgaa

SEQ ID NO: 4 - DNA, 1044 bp, Synthetic construct
  source 1..1044: mol_type = other DNA
atgaaaaacagtaagttaaaggcggttgattttttctgtggcggaggcggaatgagctatggaatgtaatccgctttagccgggatagattacgaacttagctgcaaagaaacatatgaagccaatataaatgccaaatttatacaggctgatatttttgaattgcagcctgaaaccttggaaaaagaactcggcttaaaaaagaatgatgatgatttgattcttataggttgtagtccttgtcaatattggagtgttattcaaacagacaaaagaaaatcggaaaaatccaagagtttgcttttggagtttcagcggtttgtagaatatttcaatcccggatatgttgttgtagagaacgtaccgggaattctgagcagaatgaaagaaagcgggcttgataatttcataaagttattggaagaaaaaggatttaccgtccatttcggtattcacaatacggctgattacggaattccccaaagccgtaaaagatttacgttaattgcaaacagaataaccaaaaaaaagctggaaccagtcaagtattcgggcaaacggcttacgatacgcgatgttttgggaatggaaaacggctttcccaaaattatggcaggacatcaagacgagacggattttatgcatagttgtgcgggattatccgatatcaatttgaaacgattggctttgatacctaaaaacggaggaaaccgtttggcttttgcgcatattcccgaattacagctggaatgttttatcggaaaagacaatagttttaaagacacgttcggtaggctatggtgggataagcccgcacctaccataacgaccaaattctttagcatttccaacgggcggtttgcccatcctgaagaagatagggcgttatccctgcgagaaggtgcaacattgcaatcgttccctcgtaactatgtctttaaagcgggcagtagggacaagatagcccgtttgattggtaatgccgttcctccgatgtatgcggaaaaaataggcagggcaattgttgataatatcgaatgttag

SEQ ID NO: 5 - DNA, 948 bp, Synthetic construct
  source 1..948: mol_type = other DNA
atgtacaaaacaatagatttattttcaggcattggcggaatacgcctaggctttgaaaaatacgggtgcaccaatgtattctcatcggaatgggacaaatatgcccgacaagtgtatgaagcaaactttggagaaaaaccttttggcgacattaacggcatcgatccttcagatattcccgatcatgatattctgcttgcgggatttccgtgccaacctttcagtatcgcaggtaagggattgggatttgaagatacgcgcggaacattgtttttcaatattgccgaaatcctgaaaaccaaacagccgaaggcttttcttctggaaaacgttaagcggctgactacgcacgattcgggcaggactttccgtattgttttagaaacccttaaacagcttggttatacggtttattttaaggttttaaatactttggatttcggattgccgcaaaaacgcgagagaatttatattgtgggtttttcagacaacatccctttttactttcccgaacccataaaccaatatcggccattgggcgaactgctggaaaacgatcgagatgtcgaaccaagctattttttatcagataccttgaaacaaaaacgtttggcagccttaaaaaaagctccgcccaccccatcaatttggcatgaaaatattggcgggaatgtttccgcacttccatattcctgtgccttaagggccgggggaagttacaattatttggtggtaaacggagtaagaaggctaacaggaagggaaatgttaagattacaaggttttcccgatgattttgaaatcaatatcccctactcgcaagtcaggaaagtcgccggaaattctgtatccgtccctgtaatagaagccatcgcggaaaatatgctcgcttctctttccggcaaggtcgaacaaaaagggcaattggatttattggaggcaggatga

SEQ ID NO: 6 - DNA, 1272 bp, Synthetic construct
  source 1..1272: mol_type = other DNA
atgcaacaaattaaatttattgacttattttccggaatgagtggcatcaggaaaggattcgaacaagcctgtcggaaacagtcggttgcttgcgaatgtgtttttacttcagaaatcaaaccggcagctttggaagtattgaagcaaaactaccctgatgaagtgccgtatggagatataacgaaaattgaaacaggggatattcccgattttgacatcctgttggcaggcttcccttgtcaggctttttctttcgccggaaaaagattaggctttgaagatacacggggaacgcttttctttgatgtggcaaggattttaaaggcaaaaaaaccaaaaggttttattttggaaaatgtggaaggattggtgacacacgatagaaaagattcgacacaaaaaatagggcgcaccctgaccgttattttggaaaccttggaagcattgggctactatgtttcttggaaagttttaaatgcaaaagatttcggcattccccaaaacaggaagcgcatttatctgacaggcagtttgaaatccaaacccgacttatctttcgaaacaagcccaagtccgaaattaaaaaatattttggaatcgggactgcctactgaaagcagccctttcatcaaaaaattgctaaaaaaatttcccccgtccgaactgtacggaaaatcagtaaaagacaaacggggagggaaaaacaatattcacagttgggatattgaattaaaaggcgcagtaaccgaagaagagaagcaattgttaaatatccttctaaaagaacggaggaaaaaaaaatgggcttcagaaatcggcatagattggatggatgggatgcctttgacaaaagcgcaaatttcaactttctataaacaccccgatctccaaaatattttggacagcctgacagacaaaggctatttggttttagagcatccgaaacaaaaaattggcgggcaaagaatcaaagatgaatccctgcccaaaggctacaatattgtttcaggtaaaaaatcttttgaaatcaataaaatattagatccaaacgatgttgcgccaaccttggttgcaatggatatggagcaccttttcgtcgttgacaacggcggtttgcgtacacttaccggaaaagaagggttacgcttattcggctatccggacgattattcgtttgatattcccaaaaaagacagatgcgatttattgggtaataccgttgccgtccctgtgattaaggcggtatctgaaagacttctgcatactttataa

SEQ ID NO: 7 - DNA, 1903 bp, Synthetic construct
  source 1..1903: mol_type = other DNA
aaaaacagtaagttaaaggcggttgattttttctgtggcggaggcggaatgagctatggaatgtaatccgctttagccgggatagattacgaacttagctgcaaagaaacatatgaagccaatataaatgccaaatttatacaggctgatatttttgaattgcagcctgaaaccttggaaaaagaactcggcttaaaaaagaatgatgatgatttgattcttataggttgtagtccttgtcaatattggagtgttattcaaacagacaaaagaaaatcggaaaaatccaagagtttgcttttggagtttcagcggtttgtagaatatttcaatcccggatatgttgttgtagagaacgtaccgggaattctgagcagaatgaaagaaagcgggcttgataatttcataaagttattggaagaaaaaggatttaccgtccatttcggtattcacaatacggctgattacggaattccccaaagccgtaaaagatttacgttaattgcaaacagaataaccaaaaaaaagctggaaccagtcaagtattcgggcaaacggcttacgatacgcgatgttttgggaatggaaaacggctttcccaaaattatggcaggacatcaagacgagacggattttatgcatagttgtgcgggattatccgatatcaatttgaaacgattggctttgatacctaaaaacggaggaaaccgtttggcttttgcgcatattcccgaattacagctggaatgttttatcggaaaagacaatagttttaaagacacgttcggtaggctatggtgggataagcccgcacctaccataacgaccaaattctttagcatttccaacgggcggtttgcccatcctgaagaagatagggcgttatccctgcgagaaggtgcaacattgcaatcgttccctcgtaactatgtctttaaagcgggcagtagggacaagatagcccgtttgattggtaatgccgttcctccgatgtatgcgcaggcattggcggaatacgcctaggctttgaaaaatacgggtgcaccaatgtattctcatcggaatgggacaaatatgcccgacaagtgtatgaagcaaactttggagaaaaaccttttggcgacattaacggcatcgatccttcagatattcccgatcatgatattctgcttgcgggatttccgtgccaacctttcagtatcgcaggtaagggattgggatttgaagatacgcgcggaacattgtttttcaatattgccgaaatcctgaaaaccaaacagccgaaggcttttcttctggaaaacgttaagcggctgactacgcacgattcgggcaggactttccgtattgttttagaaacccttaaacagcttggttatacggtttattttaaggttttaaatactttggatttcggattgccgcaaaaacgcgagagaatttatattgtgggtttttcagacaacatccctttttactttcccgaacccataaaccaatatcggccattgggcgaactgctggaaaacgatcgagatgtcgaaccaagctattttttatcagataccttgaaacaaaaacgtttggcagccttaaaaaaagctccgcccaccccatcaatttggcatgaaaatattggcgggaatgtttccgcacttccatattcctgtgccttaagggccgggggaagttacaattatttggtggtaaacggagtaagaaggctaacaggaagggaaatgttaagattacaaggttttcccgatgattttgaaatcaatatcccctactcgcaagtcaggaaagtcgccggaaattctgtatccgtccctgtaatagaagccatcgcggaaaatatgctcgcttctctttccggcaaggtcgaacaaaaagggcaattgga

SEQ ID NO: 8 - DNA, 2172 bp, Synthetic construct
  source 1..2172: mol_type = other DNA
atgacacgcttcaaatactccctgctttttgccgccctgctacccgtgtacgcgcaggccgatgtttctgtttcagacgaccccaaaccgcaggaaagcaccgaattgccgaccatcaccgttaccgccgaccgcaccgcgagttccaacgacggctacaccgtttccggcacgcacaccccgttcgggctgcccatgaccctgcgcgaaatcccgcagagcgtcagcgtcatcacatcgcaacaaatgcgcgaccaaaacatcaaaacgctcgaccgcgccctgttgcaggcgaccggcaccagccgccagatttacggctccgaccgcgcgggctacaactacctgttcgcgcgcggcagccgcatcgccaactaccaaatcaacggcatccccgttgccgacgcgctggccgatacgggcaatgccaacaccgccgcctatgagcgcgtggaagtcgtgcgcggcgtggcggggctgccggacggcacgggcgagccttctgccaccgtcaatctggtacgcaaacacccgacccgcaagccattgtttgaagtccgcgccgaagccggcaaccgcaaacatttcgggctgggcgcggacgtatcgggcagcctgaacgccgaaggcacgctgcgcggccgcctggtttccaccttcggacgcggcgactcgtggcggcagctcgaacgcagccgcgatgccgaactctacggcattttggaatacgacatcgcaccgcaaacccgcgtccacgcaggcatggactaccagcaggcgaaagaaaccgcagacgcgccgctcagctacgccgtgtacgacagccaaggttatgccaccgccttcggcccaaaagacaaccccgccacaaattggtcgaacagccgcaaccgtgcgctcaacctgttcgccggcatagaacaccgcttcaaccaagactggaaactcaaagccgaatacgactacacccgtagccgcttccgccagccctacggtgtggcaggcgtactttccatcgaccacagcactgccgccaccgacctgattcccggttattggcacgccgatccgcgcacccacagcgccagcatgtcattgaccggcaaataccgcctgttcggccgcgagcacgatttaatcgcgggtatcaacggctacaaatacgccagcaacaaatacggcgaacgcagcatcattcccaacgccattcccaacgcctacgaattttcccgcacgggcgcctatccgcagccatcatcgtttgcccaaaccatcccgcaatacgacaccaggcggcaaatcggcggctatctcgccacccgtttccgcgccgccgacaacctttcgctgatactcggcggcagatacagccgctaccgcgcaggcagctacaacagccgcacacaaggcatgacctatgtgtccgccaaccgtttcaccccctacacaggcatcgtgttcgatctgaccggcaacctgtcgctttacggctcgtacagcagcctgttcgtcccgcaattgcaaaaagacgaacacggcagctacctgaaacccgtaaccggcaacaatctggaagccgacatcaaaggcgaatggcttgaagggcgtctgaacgcatccgccgccgtgtaccgcgcccgtaaaaacaacctcgccaccgcagcaggacgcgaccagagcggcaacacctactaccgcgccgccaaccaagccaaaacccacggctgggaaatcgaagtcggcggccgcatcacgcccgaatggcagatacaggcaggctacagccaaagcaaaccccgcgaccaagacggcagccgcccgaaccccgacagcgtacccgaacgcagcttcaaactcttcaccgcctaccacttagcccccgaagcccccagcggccggaccatcggtgcgggtgtgcagggcgaaacccacaccgacccagccgcgctccacatccccaatcccgccgccaaagcccgcgccgtcgccaacagccgccagaaagcctacgccgtcgccgacatcatggcgcgttaccgcttcaatccgcgcaccgaactgtcgctgaacgtggacaacctgttcaacaaacactaccgcacccagcccgaccgccacagctacggcgcactgcggacagtgaacgcggcgtttacctatcggtttaaataa

SEQ ID NO: 9 - DNA, 2718 bp, Synthetic construct
  source 1..2718: mol_type = other DNA
atgctatatcatcatagttatgcagaagatgcagggcgcgcgggcagcgaggcgcagatacaggttttggaagacgtgcacgtcaaggcgaagcgcgtaccgaaagacaaaaaagtgtttaccgatgcgcgtgccgtatcgacccgtcaggatatattcaaatccggcgaaaacctcgacaacatcgtacgtagcatacccggtgcgtttacacagcaagataaaagctcgggcattgtgtctttgaatattcgcggcgacagcgggttcgggcgggtcaatacgatggtggacggcatcacgcagaccttttattcgacttctaccgatgcgggcagggcaggcggttcatctcaattcggtgcatctgtcgacagcaattttattgccggactggatgtcgtcaaaggcagcttcagcggctcggcaggcatcaacagccttgccggttcggcgaatctgcggactttaggcgtggatgacgtcgttcagggcaataatacctacggcctgctgctaaaaggtctgaccggcaccaattcaaacaaaggcaatgcgatggcggcgataggtgcgcgcaaatggctggaaagcggagcgtctgccggtgtgctttacgggcacagcaggcgcggcgtggcgcaaaattaccgcgtgggcggcggcgggcagcacatcggaaattttggtgaagaatatctggaacggcgcaaacagcaatattttgtacaagagggtggtttgaaattcaatgccggcagcggaaaatgggaacgggatttgcaaaggcaatactggaaaacaaagtggtataaaaaatacgaagacccccaagaactgcaaaaatacatcgaagagcatgataaaagctggcgggaaaacctggcgccgcaatacgacatcacccccatcgatccgtccggcctgaagcagcagtcggcaggcaatctgtttaaattggaatacgacggcgtattcaataaatacacggcgcaatttcgcgatttaaacaccaaaatcggcagccgcaaaatcatcaaccgcaattatcaattcaattacggtttgtctttgaacccgtataccaacctcaatctgaccgcagcctacaattcgggcaggcagaaatatccgaaaggggcgaagtttacaggctgggggcttttaaaagattttgaaacctacaacaacgcgaaaatcctcgacctcaacaacaccgccaccttccggctgccccgcgaaaccgagttgcaaaccactttgggcttcaattatttccacaacgaatacggcaaaaaccgctttcctgaagaattggggctgtttttcgacggtccggatcaggacaacgggctttattcctatttggggcggtttaagggcgataaagggctgctgccccaaaaatcaaccatcgtccaaccggccggcagtcaatatttcaacacgttctacttcgatgccgcgctcaaaaaagacatttaccgcttaaactacagcaccaatgcaatcaactaccgtttcggcggcgaatatacgggctattacggctcggaaagcgaatttaagcgggcattcggagaaaactcgccggcatacaaggaacattgcgacccgagctgcgggctttatgaacccgtattgaaaaaatacggcaaaaagcgcgccaacaaccattcggtcagcattagtgcggacttcggcgattatttcatgccgttcgccggctattcgcgcacacaccgtatgcccaacatccaagaaatgtatttttcccaaatcggcgactccggcgttcacaccgccttaaaaccagagcgcgcaaacacttggcaatttggcttcaatacctataaaaaaggattgttaaaacaagatgatatattaggattgaaactggtcggctaccgcagccgcattgacaactacatccacaacgtttacgggaaatggtgggatttgaacggggatattccgagctgggtcggcagcaccgggcttgcctacaccatccgacaccgcaatttcaaagacaaagtgcacaaacacggttttgagctggagctgaattacgattatgggcgttttttcaccaacctttcttacgcctatcaaaaaagcacgcaaccgaccaatttcagcgatgcgagcgaatcgcccaacaatgcctccaaagaagaccaactcaaacaaggttatgggctgagcagggtttccgccctgccgcgagattacggacgtttggaagtcggtacgcgctggttgggcaacaaactgactttgggcggcgcgatgcgctatttcggcaagagcatccgcgcgacggctgaagaacgctatatcgacggcaccaacgggggaaataccagcaatgtccggcaactgggcaagcgttccatcaaacaaaccgaaacccttgcccgacagcctttgatttttgatttttacgccgcttacgagccgaagaaaaaccttattttccgcgccgaagtcaaaaacctgttcgacaggcgttatatcgatccgctcgatgcgggcaatgatgcggcaacgcagcgttattacagctcgttcgacccgaaagacaaggacgaagacgtaacgtgtaatgctgataaaacgttgtgcaacggcaaatacggcggcacaagcaaaagcgtattgaccaatttcgcacgcggacgcaccttcttgatgacgatgagctacaagttttaa

SEQ ID NO: 10 - DNA, 4599 bp, Synthetic construct
  source 1..4599: mol_type = other DNA
atgaaagccaaacgttttaaaattaacgccatatccttatccatctttcttgcctatgcccttacgccatactcagaagcggcattggtgagagacgatgtcgattatcaaatattccgtgactttgcagaaaacaaaggcaaattttttgtcggcgcaaccgatttatcagtgaaaaacaaacgaggtcaaaacatcggcaacgcattatccaacgtaccgatgattgattttagcgttgcagatgtcaacaaacgcatagcaacggtagtcgatccccaatatgccgtcagcgtcaaacacgccaaagcagaagtccatactttttattacggccaatacaacggtcataatgatgtagccgacaaagaaaatgaataccgcgttgtcgaacaaaataactatgaaccccataaagcttggggtgcgagcaatttaggccgcctcgaagattataatatggcgcgttttaataagtttgtaaccgaagtggcaccgattgcaccaactgatgccggcggcggattggatacctacaaagataaaaaccgtttttcctcttttgtcagaatcggtgcgggcaggcaattagtttatgaaaaaggggtttatcatcaagaaggaaatgaaaaaggctacgatttgcgcgatctttcacaagcctatcgttatgccattgcaggtacgccttataaagatattaatattgaccaaacaatgaataccgaaggtttgatcggctttggtaatcataataaacagtattcggcagaagaacttaaacaagcactttcgcaagatgcgttaacaaattacggcgtgttgggcgatagcggctctccactatttgcttttgacaaacaaaaaaatcaatgggtctttttgggaacttacgattattgggcaggttacggaaaaaaatcatggcaagaatggaatatctataaaaaagaatttgcagataaaatcaaacaacacgataacgccggcaccgtcaaaggtaatggagaacatcattggaaaaccacgggtacaaacagccatatcggttcgacagcggtaaggcttgccaacaatgaaggagatgcgaacaacggacaaaatgttacctttgaagacaacggcactttggtattgaatcaaaacatcaaccaaggcgcgggcggcctgtttttcaaaggcgattacacagtcaaaggcgcaaataatgacatcacttggttaggtgcggggattgatgttgccgacggcaaaaaagtcgtttggcaagtcaaaaatccgaatggcgacagattggcaaaaatcggcaaaggcactttggaaataaacggcacaggcgttaaccaagggcaattaaaggtcggcgacggtacggttattctgaatcaaaaagccgatgccgacaaaaaagttcaggctttctcccaagtcggcattgtcagcggacgcggtacattggtattaaatagttcaaatcagattaatcccgataacctatatttcggtttccgtggcggtcgtttggatgccaatggcaatgacttgacttttgaacacatccgcaacgtggatgaaggcgcgcgcattgtcaaccacaacacagaccacgcctccacaatcacactaacgggtaaatctttaattaccaatccgaacagcctttcggtacatagcatacaaaatgactatgatgaagatgattatagttattattaccgaccaaggcgacccatcccacaaggcaaagatctttattacaaaaactatcgttattacgccctaaaatccggcggcagcgtgaacgcaccgatgcccgagaacggtgtagcagaaaataacgactgggtctttatgggatatacgcaagaagaagccaagaaaaacgcgatgaaccacaaaaacaatcagcgtattagcggttttagcggtttctttggcgaagaaaacggaaaaggacataacggcgcattaaaccttaatttcaatggcaaaagcgcgcaaaaccgtttcttgttaacaggcggcgccaatttaaacggaaaaataagcgtaactcaaggcaatgtcttgttatcaggtcgtccaacaccacacgcaagagattttgtgaacaaatcttcagcccgaaaagacgcacatttctccaaaaacaatgaagtcgtatttgaagacgactggataaaccgcacattcaaagccgcagaaattgcggttaaccaatccgcatcattctcttccggaagaaatgtatccgacatcaccgccaacatcaccgcaacagacaacgccaaagtaaatttgggttacaaaaacggcgatgaggtttgcgtgcgctcggactataccggttacgttacctgcaacacaggcaacttatccgataaggctttaaacagctttgatgcgacacggattaacggcaatgtgaatttgaatcagaatgcggcattggtcttgggcaaggctgcattatgggggaaaattcaaggacaaggaaacagccgtgtcagcctaaaccaacatagcaaatggcatttgaccggcgacagccaagtacacaatctgtcattggcggatagccatattcatttgaacaacgcttccgatgcgcaaagtgcaaataaataccacacgatcaaaatcaatcatttatccggtaacgggcattttcattatctgacggacttggcgaaaaatcttggggataaagtgcttgtgaaggaatccgcatccggccattatcagctccatgttcaaaataaaacaggcgaacctaatcaggaagggctggatctctttgatgcatcatccgtacaagaccgctcccgcctttttgtttccttggcaaatcattacgtcgatttaggcgcattgcgttatacaatcaaaacagaaaacggtattacccggttgtacaatccttatgccgggaaccgccgcccagtcaagccggccccgtctcctgccgcaaacacggcttctcaagcacaaaaggcaacacaaacggacggtgcacaaattgccaagcctcaaaatatcgtcgtcgcaccgcctagcccgcaggcaaatcaagccgaagaagccctccgccaacaagcaaaagcggagcaagtgaagcgtcagcaagcagcagaagcagaaaaagttgcacgtcaaaaagacgaagaggcaaaacgcaaagcagccgaaattgctcgtcagcaggaagaagcacgaaaagctgcagagttagccgccaaacaaaaggcggaagcagagcgtaaagccagagagttggcaagacaaaaagcagaagaggcaagtcatcaagctaatgccaaaccaaaacgtcgtagacgtcgggctatattacctagacctcctgcgccagtattttcattggatgattatgatgcaaaagacaatagtgaatcatcaataggtaatttagctcgtgtaatacctagaatgggaagggagttaattaatgattatgaagaaatccccttggaggagttggaagatgaagcggaagaagaacgtcgccaagcaacgcaattccaacccaaaagtcgtaaccgtagagctatatcatcggaaccatcatctgatgaagatgcatctgaatcggtttccacatcagacaaacaccctcaagataatacggaacttcatgaaaaagttgagacggcgggtttacaaccaagagccgcgcagccgcgaacccaagccgccgcgcaagccgatgcagtcagcaccaatactaactcggctttatctgacgcaatggcaagcacgcaatctatcttgttggatacaggtgcttcattaacacggcacattgcacaaaaatcacgcgctgatgccgaaaaaaacagtgtttggatgtcaaacaccggttatggccgtgattatgcttccgcacaatatcgccggtttagttcgaaacgcacgcaaacacaaatcggcattgaccgcagcttgtccgaaaatatgcagataggcggagtattgacttactctgacagtcagcatacttttgatcaggcgggcggcaaaaatacttttgtgcaagccaacctttatggtaagtattatttaaatgatgcttggtatgtggccggcgatattggtgcgggcagcttgagaagccggttacaaacgcagcaaaaagcaaactttaaccgaacaagcatccaaaccggccttactttgggcaatacgctgaaaatcaatcaattcgagattgtccctagtgcgggtatccgttacagccgcctgtcatctgcagattacaagttgggtgacgacagtgttaaagtaagttctatggcagtgaaaacactaacggccggactggattttgcttatcggtttaaagtcggcaaccttaccgtaaaacccttgttatctgcagcttactttgccaattatggcaaaggcggcgtgaatgtgggcggtaaatccttcgcctataaagcagataatcaacagcaatattcagcaggcgccgcgttactgtaccgtaatgttacattaaacgtaaatggcagtattacaaaaggaaaacaattggaaaaacaaaaatccggacaaattaaaatacagattcgtttctaa

SEQ ID NO: 11 - DNA, 8284 bp, Synthetic construct
  source 1..8284: mol_type = other DNA
caggccgatgtttctgtttcagacgaccccaaaccgcaggaaagcaccgaattgccgaccatcaccgttaccgccgaccgcaccgcgagttccaacgacggctacaccgtttccggcacgcacaccccgttcgggctgcccatgaccctgcgcgaaatcccgcagagcgtcagcgtcatcacatcgcaacaaatgcgcgaccaaaacatcaaaacgctcgaccgcgccctgttgcaggcgaccggcaccagccgccagatttacggctccgaccgcgcgggctacaactacctgttcgcgcgcggcagccgcatcgccaactaccaaatcaacggcatccccgttgccgacgcgctggccgatacgggcaatgccaacaccgccgcctatgagcgcgtggaagtcgtgcgcggcgtggcggggctgccggacggcacgggcgagccttctgccaccgtcaatctggtacgcaaacacccgacccgcaagccattgtttgaagtccgcgccgaagccggcaaccgcaaacatttcgggctgggcgcggacgtatcgggcagcctgaacgccgaaggcacgctgcgcggccgcctggtttccaccttcggacgcggcgactcgtggcggcagctcgaacgcagccgcgatgccgaactctacggcattttggaatacgacatcgcaccgcaaacccgcgtccacgcaggcatggactaccagcaggcgaaagaaaccgcagacgcgccgctcagctacgccgtgtacgacagccaaggttatgccaccgccttcggcccaaaagacaaccccgccacaaattggtcgaacagccgcaaccgtgcgctcaacctgttcgccggcatagaacaccgcttcaaccaagactggaaactcaaagccgaatacgactacacccgtagccgcttccgccagccctacggtgtggcaggcgtactttccatcgaccacagcactgccgccaccgacctgattcccggttattggcacgccgatccgcgcacccacagcgccagcatgtcattgaccggcaaataccgcctgttcggccgcgagcacgatttaatcgcgggtatcaacggctacaaatacgccagcaacaaatacggcgaacgcagcatcattcccaacgccattcccaacgcctacgaattttcccgcacgggcgcctatccgcagccatcatcgtttgcccaaaccatcccgcaatacgacaccaggcggcaaatcggcggctatctcgccacccgtttccgcgccgccgacaacctttcgctgatactcggcggcagatacagccgctaccgcgcaggcagctacaacagccgcacacaaggcatgacctatgtgtccgccaaccgtttcaccccctacacaggcatcgtgttcgatctgaccggcaacctgtcgctttacggctcgtacagcagcctgttcgtcccgcaattgcaaaaagacgaacacggcagctacctgaaacccgtaaccggcaacaatctggaagccgacatcaaaggcgaatggcttgaagggcgtctgaacgcatccgccgccgtgtaccgcgcccgtaaaaacaacctcgccaccgcagcaggacgcgaccagagcggcaacacctactaccgcgccgccaaccaagccaaaacccacggctgggaaatcgaagtcggcggccgcatcacgcccgaatggcagatacaggcaggctacagccaaagcaaaccccgcgaccaagacggcagccgcccgaaccccgacagcgtacccgaacgcagcttcaaactcttcaccgcctaccacttagcccccgaagcccccagcggccggaccatcggtgcgggtgtgcagggcgaaacccacaccgacccagccgcgctccacatccccaatcccgccgccaaagcccgcgccgtcgccaacagccgccagaaagcctacgccgtcgccgacatcatggcgcgttaccgcttcaatccgcgcaccgaactgtcgctgaacgtggacaacctgttcagggtcaatacgatggtggacggcatcacgcagaccttttattcgacttctaccgatgcgggcagggcaggcggttcatctcaattcggtgcatctgtcgacagcaattttattgccggactggatgtcgtcaaaggcagcttcagcggctcggcaggcatcaacagccttgccggttcggcgaatctgcggactttaggcgtggatgacgtcgttcagggcaataatacctacggcctgctgctaaaaggtctgaccggcaccaattcaaacaaaggcaatgcgatggcggcgataggtgcgcgcaaatggctggaaagcggagcgtctgccggtgtgctttacgggcacagcaggcgcggcgtggcgcaaaattaccgcgtgggcggcggcgggcagcacatcggaaattttggtgaagaatatctggaacggcgcaaacagcaatattttgtacaagagggtggtttgaaattcaatgccggcagcggaaaatgggaacgggatttgcaaaggcaatactggaaaacaaagtggtataaaaaatacgaagacccccaagaactgcaaaaatacatcgaagagcatgataaaagctggcgggaaaacctggcgccgcaatacgacatcacccccatcgatccgtccggcctgaagcagcagtcggcaggcaatctgtttaaattggaatacgacggcgtattcaataaatacacggcgcaatttcgcgatttaaacaccaaaatcggcagccgcaaaatcatcaaccgcaattatcaattcaattacggtttgtctttgaacccgtataccaacctcaatctgaccgcagcctacaattcgggcaggcagaaatatccgaaaggggcgaagtttacaggctgggggcttttaaaagattttgaaacctacaacaacgcgaaaatcctcgacctcaacaacaccgccaccttccggctgccccgcgaaaccgagttgcaaaccactttgggcttcaattatttccacaacgaatacggcaaaaaccgctttcctgaagaattggggctgtttttcgacggtccggatcaggacaacgggctttattcctatttggggcggtttaagggcgataaagggctgctgccccaaaaatcaaccatcgtccaaccggccggcagtcaatatttcaacacgttctacttcgatgccgcgctcaaaaaagacatttaccgcttaaactacagcaccaatgcaatcaactaccgtttcggcggcgaatatacgggctattacggctcggaaagcgaatttaagcgggcattcggagaaaactcgccggcatacaaggaacattgcgacccgagctgcgggctttatgaacccgtattgaaaaaatacggcaaaaagcgcgccaacaaccattcggtcagcattagtgcggacttcggcgattatttcatgccgttcgccggctattcgcgcacacaccgtatgcccaacatccaagaaatgtatttttcccaaatcggcgactccggcgttcacaccgccttaaaaccagagcgcgcaaacacttggcaatttggcttcaatacctataaaaaaggattgttaaaacaagatgatatattaggattgaaactggtcggctaccgcagccgcattgacaactacatccacaacgtttacgggaaatggtgggatttgaacggggatattccgagctgggtcggcagcaccgggcttgcctacaccatccgacaccgcaatttcaaagacaaagtgcacaaacacggttttgagctggagctgaattacgattatgggcgttttttcaccaacctttcttacgcctatcaaaaaagcacgcaaccgaccaatttcagcgatgcgagcgaatcgcccaacaatgcctccaaagaagaccaactcaaacaaggttatgggctgagcagggtttccgccctgccgcgagattacggacgtttggaagtcggtacgcgctggttgggcaacaaactgactttgggcggcgcgatgcgctatttcggcaagagcatccgcgcgacggctgaagaacgctatatcgacggcaccaacgggggaaataccagcaatgtccggcaactgggcaagcgttccatcaaacaaaccgaaaccttggcaccgattgcaccaactgatgccggcggcggattggatacctacaaagataaaaaccgtttttcctcttttgtcagaatcggtgcgggcaggcaattagtttatgaaaaaggggtttatcatcaagaaggaaatgaaaaaggctacgatttgcgcgatctttcacaagcctatcgttatgccattgcaggtacgccttataaagatattaatattgaccaaacaatgaataccgaaggtttgatcggctttggtaatcataataaacagtattcggcagaagaacttaaacaagcactttcgcaagatgcgttaacaaattacggcgtgttgggcgatagcggctctccactatttgcttttgacaaacaaaaaaatcaatgggtctttttgggaacttacgattattgggcaggttacggaaaaaaatcatggcaagaatggaatatctataaaaaagaatttgcagataaaatcaaacaacacgataacgccggcaccgtcaaaggtaatggagaacatcattggaaaaccacgggtacaaacagccatatcggttcgacagcggtaaggcttgccaacaatgaaggagatgcgaacaacggacaaaatgttacctttgaagacaacggcactttggtattgaatcaaaacatcaaccaaggcgcgggcggcctgtttttcaaaggcgattacacagtcaaaggcgcaaataatgacatcacttggttaggtgcggggattgatgttgccgacggcaaaaaagtcgtttggcaagtcaaaaatccgaatggcgacagattggcaaaaatcggcaaaggcactttggaaataaacggcacaggcgttaaccaagggcaattaaaggtcggcgacggtacggttattctgaatcaaaaagccgatgccgacaaaaaagttcaggctttctcccaagtcggcattgtcagcggacgcggtacattggtattaaatagttcaaatcagattaatcccgataacctatatttcggtttccgtggcggtcgtttggatgccaatggcaatgacttgacttttgaacacatccgcaacgtggatgaaggcgcgcgcattgtcaaccacaacacagaccacgcctccacaatcacactaacgggtaaatctttaattaccaatccgaacagcctttcggtacatagcatacaaaatgactatgatgaagatgattatagttattattaccgaccaaggcgacccatcccacaaggcaaagatctttattacaaaaactatcgttattacgccctaaaatccggcggcagcgtgaacgcaccgatgcccgagaacggtgtagcagaaaataacgactgggtctttatgggatatacgcaagaagaagccaagaaaaacgcgatgaaccacaaaaacaatcagcgtattagcggttttagcggtttctttggcgaagaaaacggaaaaggacataacggcgcattaaaccttaatttcaatggcaaaagcgcgcaaaaccgtttcttgttaacaggcggcgccaatttaaacggaaaaataagcgtaactcaaggcaatgtcttgttatcaggtcgtccaacaccacacgcaagagattttgtgaacaaatcttcagcccgaaaagacgcacatttctccaaaaacaatgaagtcgtatttgaagacgactggataaaccgcacattcaaagccgcagaaattgcggttaaccaatccgcatcattctcttccggaagaaatgtatccgacatcaccgccaacatcaccgcaacagacaacgccaaagtaaatttgggttacaaaaacggcgatgaggtttgcgtgcgctcggactataccggttacgttacctgcaacacaggcaacttatccgataaggctttaaacagctttgatgcgacacggattaacggcaatgtgaatttgaatcagaatgcggcattggtcttgggcaaggctgcattatgggggaaaattcaaggacaaggaaacagccgtgtcagcctaaaccaacatagcaaatggcatttgaccggcgacagccaagtacacaatctgtcattggcggatagccatattcatttgaacaacgcttccgatgcgcaaagtgcaaataaataccacacgatcaaaatcaatcatttatccggtaacgggcattttcattatctgacggacttggcgaaaaatcttggggataaagtgcttgtgaaggaatccgcatccggccattatcagctccatgttcaaaataaaacaggcgaacctaatcaggaagggctggatctctttgatgcatcatccgtacaagaccgctcccgcctttttgtttccttggcaaatcattacgtcgatttaggcgcattgcgttatacaatcaaaacagaaaacggtattacccggttgtacaatccttatgccgggaaccgccgcccagtcaagccggccccgtctcctgccgcaaacacggcttctcaagcacaaaaggcaacacaaacggacggtgcacaaattgccaagcctcaaaatatcgtcgtcgcaccgcctagcccgcaggcaaatcaagccgaagaagccctccgccaacaagcaaaagcggagcaagtgaagcgtcagcaagcagcagaagcagaaaaagttgcacgtcaaaaagacgaagaggcaaaacgcaaagcagccgaaattgctcgtcagcaggaagaagcacgaaaagctgcagagttagccgccaaacaaaaggcggaagcagagcgtaaagccagagagttggcaagacaaaaagcagaagaggcaagtcatcaagctaatgccaaaccaaaacgtcgtagacgtcgggctatattacctagacctcctgcgccagtattttcattggatgattatgatgcaaaagacaatagtgaatcatcaataggtaatttagctcgtgtaatacctagaatgggaagggagttaattaatgattatgaagaaatccccttggaggagttggaagatgaagcggaagaagaacgtcgccaagcaacgcaattccaacccaaaagtcgtaaccgtagagctatatcatcggaaccatcatctgatgaagatgcatctgaatcggtttccacatcagacaaacaccctcaagataatacggaacttcatgaaaaagttgagacggcgggtttacaaccaagagccgcgcagccgcgaacccaagccgccgcgcaagccgatgcagtcagcaccaatactaactcggctttatctgacgcaatggcaagcacgcaatctatcttgttggatacaggtgcttcattaacacggcacattgcacaaaaatcacgcgctgatgccgaaaaaaacagtgtttggatgtcaaacaccggttatggccgtgattatgcttccgcacaatatcgccggtttagttcgaaacgcacgcaaacacaaatcggcattgaccgcagcttgtccgaaaatatgcagataggcggagtattgacttactctgacagtcagcatacttttgatcaggcgggcggcaaaaatacttttgtgcaagccaacctttatggtaagtattatttaaatgatgcttggtatgtggccggcgatattggtgcgggcagcttgagaagccggttacaaacgcagcaaaaagcaaactttaaccgaacaagcatccaaaccggccttactttgggcaatacgctgaaaatcaatcaattcgagattgtccctagtgcgggtatccgttacagccgcctgtcatctgcagattacaagttgggtgacgacagtgttaaagtaagttctatggcagtgaaaacactaacggccggactggattttgcttatcggtttaaagtcggcaaccttaccgtaaaacccttgttatctgcagcttactttgccaattatggcaaaggcggcgtgaatgtgggcggtaaatccttcgcctataaagcagataatcaacagcaatattcagcaggcgccgcgttactgtaccgtaatgttacattaaacgtaaatggcagtattacaaaaggaaaacaattggaaaaacaaaaatccggacaaattaaaatacagattcgtttctaaa